(19) United States
(12) Patent Application Publication
(10) Pub. No.: US 2026/0174108 A1
(43) Pub. Date: Jun. 25, 2026

(54) AN EDIBLE FOOD PRODUCT AND A MANUFACTURING METHOD THEREOF

(71) Applicant: SUPRÊME, Evry-Courcouronnes Cedex (FR)

(72) Inventors: Victor SAYOUS, Evry-Courcouronnes Cedex (FR); Victor CONVERSA MARTINEZ, Evry-Courcouronnes Cedex (FR); Phuong Anh DANG, Evry-Courcouronnes Cedex (FR)

(21) Appl. No.: 19/127,224

(22) PCT Filed: Nov. 9, 2023
(86) PCT No.: PCT/EP2023/081372

§ 371 Date: May 5, 2025

(30) Foreign Application Priority Data
Nov. 9, 2022 (EP) 22315269.5
Nov. 9, 2022 (EP) 22315270.3
Nov. 9, 2022 (EP) 22315271.1

(51) Int. Cl. A23K 20/174; A23L 13/40; A23L 33/115; A61K 31/232

(52) U.S. Cl. CPC A23K 20/174; A23L 13/424; A23L 33/115; A61K 31/232

(57) ABSTRACT

The invention relates to a method of manufacturing an edible food product comprising edible protein threads embedded in a fatty matrix, said method comprising the following steps: —Preparing a protein matrix (130), said protein matrix comprising proteins, —Preparing a fatty matrix (140), said fatty matrix comprising at least 20% in weight of fat compared to the total weight of the fatty matrix, the protein matrix and/or the fatty matrix comprising one or more texturizing molecule capable of creating a heat-resistant thread, and —Contacting (160) the protein matrix with the fatty matrix to form edible protein threads, from the protein matrix, embedded into the fatty matrix.

### FIELD OF THE INVENTION

The present invention relates to the field of food products. In particular, the invention relates to the field of meat substitutes. In particular, this invention relates to cultured cell-based meat. In particular, this invention can provide new edible food products comprising protein threads embedded into a fatty matrix. This edible product with a meat-like texture can be considered as a substitute for conventional meat.

### DESCRIPTION OF RELATED ART

From 2020 to 2050, the world population will rise by an estimated 2 billion (United Nations, 2015). Humanity will face major challenges among which is food production, including meat production. Traditional meat production is a resource-intensive process that generates a significant environmental footprint. Domesticated animals are raised in agricultural settings requiring substantial quantities of fresh water, feed, land, and other resources (Mark J Post: “Cultured meat from stem cells: Challenges and prospects” Meat science, Elsevier Science, GB, vol 92, no 3, 3 Apr. 2012, pages 297-301). Consequently, food production is already considered responsible for approximately 26% of global greenhouse gas (GHG) emissions, of which livestock & fisheries account for 31%. A reduction in global conventional meat consumption could lead to a significant reduction in emissions of greenhouse gases related to climate change (Martin & Brandão, 2017. Evaluating the environmental consequences of Swedish food consumption and dietary choices. Sustainability, 9(12), 2227), particularly if reductions are made in countries where meat consumption is high or rising (Stoll-Kleemann & Schmidt, 2017, Reducing meat consumption in developed and transition countries to counter climate change and biodiversity loss: A review of influence factors. Regional Environmental Change, 17(5), 1261-1277.; Collier et al. 2021, Identifying barriers to decreasing meat consumption and increasing acceptance of meat substitutes among Swedish consumers; Appetite 167 (2021) 105643.). Moreover, animal welfare has been a growing concern. For example, the European Union has put in place various laws concerning animal welfare since 1986. Various directives provide rules for the protection of laying hens (in 1986 and 1988), calves and pigs (in 1991), and, in 1998, the Council directive 98/58/EC on the protection of animals kept for farming purposes has established general rules concerning the protection of animals, whatever the species.

Several meat substitutes have been developed from insects, vegetable components and/or cultured animal, fungus or vegetable cells (i.e. cell technology). In particular, cell technologies are being rapidly developed to respond to new demands of consumers.

Crucial to the increase people's willingness to consume meat replacements, the product, as a substitute, should mimic the aesthetic and organoleptic qualities of meat such as size, appearance, flavor, mouthfeel and texture (Macdiarmid et al., 2016, Eating like there's no tomorrow: Public awareness of the environmental impact of food and reluctance to eat less meat as part of a sustainable diet. Appetite. 2016 Jan. 1; 96:487-493). Although the technology of texturization to improve the feel and taste of these products is continuously improving, meat analogues still differ from conventional meat in terms of mouthfeel and flavor (Samard & Ryu, 2019; A comparison of physicochemical characteristics, texture, and structure of meat analogue and meats. Journal of the Science of Food and Agriculture, 99(6), 2708-2715). Moreover, consumers also reported being unfamiliar with how to prepare meals with meat substitutes, making it more difficult and time consuming to make a satisfying, tasty meal using such products than using meat (Elzerman et al., 2013; Exploring meat substitutes: Consumer experiences and contextual factors. British Food Journal, 115(5), 700-710).

Texture has been considered as one of the most important qualities of meat analogues (Sha & Xiong, 2020. Plant protein-based alternatives of reconstructed meat: Science, technology, and challenges. Trends in Food Science & Technology. Volume 102, August 2020, Pages 51-61). Many methods have been proposed for improving the texture of meat analogues. For example, it has been proposed to combine oat and pea proteins as a practical alternative to soy and gluten proteins (Kaleda et al. 2021. Physicochemical, textural, and sensorial properties of fibrous meat analogues from oat-pea protein blends extruded at different moistures, temperatures, and screw speeds. Future Foods 4 (2021) 100092). Biosurfactant-based emulsions have also been proposed to produce 3D-printed food (Shahbazi et al. 2021. Construction of 3D printed reduced-fat meat analogue by emulsion gels. Part II: Printing performance, thermal, tribological, and dynamic sensory characterization of printed objects. Food Hydrocolloids 121 (2021) 107054). According to this study, the replacement of oil by biopolymeric surfactants is recommended to produce a fibrous, 3D-printed, low-fat meat analogue, in which the printed reduced-fat constructs presented the desired sensory profile. However, for many meat analogues a satisfying flavor-rich fat is still missing from the product. A combination of biopolymeric surfactants and hydrocolloids does not mimic the full suite of organoleptic properties typical of animal-derived meats, i.e. both texture, taste and aroma. It has also been proposed to produce an extruded food product comprising cultured animal cells (WO2022047263). However, the resulting extruded food bears little resemblance to what a consumer would expect from a meat product, especially if the desired appearance is a raw product and flavor kept within the meat piece.

Recent advances in muscle tissue engineering, such as scaffolding (Linzi Li et al. 2022. Chitosan-sodium alginate-collagen/gelatin three-dimensional edible scaffolds for building a structured model for cell cultured meat. International Journal of Biological Macromolecules. Volume 209, Part A, 1 Jun. 2022, Pages 668-679) and bioprinting combined with physicochemical stimulation, have allowed the design and manufacture of pieces of meat that increasingly emulate the natural structure and composition (Xin Guan et al. Bioprocessing technology of muscle stem cells: implications for cultured meat. Trends in Biotechnology. Volume 40, Issue 6, June 2022, Pages 721-734). However, there are regulatory issues and obvious technical obstacles to the transition to large-scale manufacturing for such technology. Moreover, there is a need for manufacturing methods allowing the production of high-fat meat and fish alternatives, such as marbled meat, salmon or tuna.

Hence, there are important needs to find a satisfactory alternative to animal slaughtering and intensive meat production means in order to produce an edible food product that has a pleasant flavor, a texture and a cooking behavior corresponding to that expected for meat. In particular, there is a need for an edible food product comprising protein threads which can mimic the conventional meat texture and in particular marbled meat. A texture expected from an animal meat before and after refined cooking is also desired to enable the consumer to cook the product in the same manner as conventional meat and to experience the same tasting quality.

### SUMMARY OF THE INVENTION

The following sets forth a simplified summary of selected aspects, embodiments and examples of the present invention for the purpose of providing a basic understanding of the invention. However, this summary does not constitute an extensive overview of all the aspects, embodiments and examples of the invention. Its sole purpose is to present selected aspects, embodiments and examples of the invention in a concise form as an introduction to the more detailed description of the aspects, embodiments and examples of the invention that follow the summary.

The invention aims to overcome the disadvantages of the prior art. In particular, the invention proposes a method of manufacturing an edible food product comprising edible protein threads embedded in a fatty matrix, said method comprising the following steps:
Preparing a protein matrix, said protein matrix comprising proteins,
Preparing a fatty matrix, said fatty matrix comprising at least 20% in weight of fat compared to the total weight of the fatty matrix,
the protein matrix and/or the fatty matrix comprising one or more texturizing molecule capable of creating a heat-resistant thread, and
Contacting the protein matrix with the fatty matrix to form edible protein threads, from the protein matrix, embedded into the fatty matrix.

Such an edible food product has a texture similar to the texture of meat notably thanks to the presence of the protein threads having a texture similar to the texture of meat fibers. In particular, the protein thread can comprise plant proteins, microbial proteins, algae proteins, animal proteins such as non-human animal protein from non-human animal cultivated cells, fungal proteins, and a combination thereof.

Moreover, as the protein threads are directly produced in a fatty matrix, this method can remove a needed following step of combining the protein threads with a fatty matrix in order to produce an edible ready-to-consume product. Thus, the method according to the invention allows to simplify and increase the yield of the manufacturing process as well as improve the yield and minimize materials losses in the production of meat alternatives. The protein threads according to the invention can exhibit a great diversity of shape and size unlike high moisture extrusion usually used for structuring plant-based products. Also, the method according to the invention can produce three-dimensional products and bigger threads containing the flavors expected by consumers.

In particular, the method according to the invention allows the production, in fewer steps, of an edible food product with a raw aspect. With such an advantage, a consumer can still cook the product in the same manner as conventional meat and experience the same organoleptic qualities. This method is scalable and allows the production of aligned threads with a higher range of fat and proteins (compared to low or high moisture extrusion) and with no oxidation while processing.

The edible food product according to the invention can be thus considered as an alternative to a conventional meat product. The edible protein threads embedded in the fatty matrix can be processed to mimic a large variety of meat fibers, including but not limited to beef meat, scallop, crab pulpit, chicken breast, duck breast, tuna meat, salmon meat, and has a matching meaty or fishy flavor.

According to other optional features of the method of manufacturing an edible food product according to the invention, it can optionally include one or more of the following characteristics alone or in combination:
the fatty matrix further comprises at least 0.25% in weight of non-human animal proteins with respect to the total wet weight of the fatty matrix.
the fatty matrix comprises triglycerides and the triglycerides of the fatty matrix comprises more than 1.5% in weight of polyunsaturated C18 fatty acids with respect to the total weight of triglycerides in the fatty matrix.
the fatty matrix comprises triglycerides and the triglycerides of the fatty matrix comprise linolenic acids for example more than 0.01% in weight of linolenic acids with respect to the total weight of triglycerides in the fatty matrix.
the protein matrix comprises at least 0.5% in weight of animal proteins being from non-human cultivated animal cells compared to the total wet weight of the protein matrix.
the protein matrix comprises at least 5% in weight of animal proteins being from non-human cultivated animal cells compared to the total weight of proteins in the protein matrix.
the protein matrix comprises at least 0.25% in weight of myofibrillar proteins compared to the total weight of proteins.
the protein matrix comprises at least 5% in weight of proteins compared to the total weight of the protein matrix.
the protein matrix comprises non-human animal proteins. In a preferred embodiment, said animal proteins are from non-human cultivated animal cells. Indeed, the presence of non-human animal proteins from cultivated animal cells introduce complex and desired flavors in the protein threads and this can be modulated according to the species of the used non-human cultivated animal cells. The non-human animal proteins can be introduced through the addition of intact cultivated cells, disrupted cultivated cells, or extracts of cultivated cells.
the texturizing molecule comprises molecule(s) capable of creating ionic bonds or covalent bonds involving said proteins. In a preferred embodiment said proteins are proteins from non-human cultivated animal cells or extracts thereof. For example, the texturizing molecule comprises a combination of a polyvalent ion and a polyelectrolyte, and/or a cross-linking molecule.
the texturizing molecule comprises a combination of a polyvalent ion and a polyelectrolyte, said polyelectrolyte being capable of complexing with the polyvalent ion to form a heat-resistant complex. For example, the protein matrix comprises said polyelectrolyte and the fatty matrix comprises said polyvalent ion. Alternatively, the protein matrix comprises said polyvalent ion and the fatty matrix comprises said polyelectrolyte. In a specific embodiment the texturizing molecule further comprises a cross-linking molecule.
the fat of the fatty matrix comprises at least 60% in weight of triglycerides compared to the total weight of the fat of the fatty matrix. Indeed, the presence of triglycerides is involved in obtaining a more appreciated meat-like texture.
the fat of the fatty matrix comprises at least 20% in weight of unsaturated fatty acids compared to the total weight of the fat of the fatty matrix. Indeed, the presence of triglycerides is involved in obtaining a more appreciated meat-like texture.
the fatty matrix has, during the contacting step, a viscosity from 100 to 50000 Pa·s, when measured at 25° C. at a shear rate of 0.1 s⁻¹
the protein matrix has, during the contacting step, a viscosity from 50 to 50000 Pa·s, when measured at 25° C. at a shear rate of 0.1 s⁻¹.
the fatty matrix and the protein matrix have, during the contacting step, viscosities such that the ratio of the viscosity of the protein matrix to the viscosity of the fatty matrix is below 1.
the preparation step of the fatty matrix further comprises a homogenizing step. The homogenization improves the properties of the edible food product. Hence, preferably, the fatty matrix comprises an emulsion, more preferably an oil in water emulsion.
the fatty matrix further comprises at least 0.25% in weight of proteins compared to the total weight of the fatty matrix.
the contacting step comprises an injection of the protein matrix into fatty matrix, for example using one or more needles, at an injection site and the contacting step comprises a movement of the injection site of the protein matrix into fatty matrix relative to the fatty matrix. Hence, during the contacting step, either the injection site is moving, the fatty matrix is moving or both are moving.
the preparation step of the fatty matrix further comprises a heat treatment of the fat to be used in the fatty matrix, preferably the fat used in the fatty matrix has been heated at a temperature of at least 50° C.
the contacting step is conducted at a temperature of at least 30° C. and the contacting step is followed by a cooling step of the protein threads embedded in the fatty matrix. The cooling step solidifies the obtained product in order to better embed the protein threads into the fatty matrix. Therefore a semi-finalized to finalized edible food product with a marbled aspect is obtained in fewer processed steps compared to methods of the state of the art.
the fatty matrix comprises:
   at least 20% in weight of fat, preferably at least 40% in weight in fat, with respect to the total wet weight of the fatty matrix; and
   at least one texturizing molecule, such as at least 0.001% in weight of the polyvalent ion with respect to the total wet weight of the fatty matrix.
the fatty matrix comprises:
   at least 20% in weight of fat, preferably at least 40% in weight in fat, with respect to the total wet weight of the fatty matrix; and
   at least two texturizing molecules including at least 0.001% in weight of the polyvalent ion with respect to the total wet weight of the fatty matrix, and at least one cross-linking molecule.
the fatty matrix comprises:
   at least 20% in weight of fat, preferably at least 40% in weight in fat, with respect to the total wet weight of the fatty matrix;
   at least one texturizing molecule; and
   proteins, said proteins comprising non-human animal proteins from non-human cultivated animal cells.

The invention also relates to an edible food product obtainable from a method according to the invention. In particular, the edible food product obtainable according to the invention comprises edible protein threads embedded in a fatty matrix. Preferably, the edible protein threads are heat-resistant threads comprising proteins and the fatty matrix comprises at least 20% in weight of fat compared to the total weight of the fatty matrix.

In particular, the proteins of the edible protein threads are selected among plant proteins, microbial proteins, algae proteins, animal proteins, fungal proteins, and a combination thereof. Preferably, the proteins of the edible protein threads comprise at least animal proteins from non-human cultivated animal cells.

The invention also relates to a system for producing an edible food product comprising edible protein threads embedded in a fatty matrix, said system comprising:
A protein matrix container capable of containing a protein matrix, said protein matrix comprising proteins,
A fatty matrix container capable of containing a fatty matrix, said fatty matrix comprising at least 20% in weight of fat compared to the total weight of the fatty matrix, the protein matrix and/or the fatty matrix comprising one or more texturizing molecule capable of creating a heat-resistant thread, and
A contacting device configured to bring in contact the protein matrix with the fatty matrix to form, from the protein matrix, edible protein threads embedded into the fatty matrix, said contacting device being arranged to inject the protein matrix into the fatty matrix in at least one injection site;
said system being configured to induce a movement of:
   the at least one injection site of the protein matrix into the fatty matrix relative to the fatty matrix, and/or
   the fatty matrix relative to the at least one injection site of the protein matrix into the fatty matrix.

Hence, the system can be configured to induce, during a contacting step, either a movement of the at least one injection site, a movement of the fatty matrix or a movement of both. Preferably, the contacting device comprises several injection sites of the protein matrix into the fatty matrix.

### BRIEF DESCRIPTION OF THE DRAWINGS

The foregoing and other objects, features and advantages of the present invention will become more apparent from the following detailed description when taken in conjunction with the accompanying drawings in which:

FIG. 1 is a schematic view of a method of producing an edible food product with meat-like texture according to an embodiment of the invention.

FIG. 2 is an illustration of a system according to the invention.

FIG. 3 is a photograph of an edible food product.

Several aspects of the present invention are disclosed with reference to flow diagrams and/or block diagrams of methods, devices and systems.

On the figures, when present, the flow diagrams and/or block diagrams show the architecture, the functionality and possible implementation of devices or systems or methods, according to several embodiments of the invention.

In some implementations, the functions associated with the box may appear in a different order than indicated in the drawings. For example, two boxes successively shown, may be performed substantially simultaneously, or boxes may sometimes be performed in the reverse order, depending on the functionality involved.

### DETAILED DESCRIPTION

A description of example embodiments of the invention follows.

The expression “protein thread” as used herein can relate to a long, thin strand of material comprising at least 2% in weight of proteins, at least 5% in weight of proteins, preferably at least 10%, more preferably at least 15% in weight of proteins compared to the wet weight of the thread. A protein thread has a diameter of at least 0.01 mm, preferably at least 0.05 mm and an aspect ratio (length/diameter) of at least 100, preferably at least 200. Preferably the protein thread has a diameter of at most 2 mm, more preferably at most 1 mm, even more preferably at most 0.5 mm. The expression “edible protein thread” as used herein can relate to a protein thread suitable for animal consumption and preferably a protein thread suitable for human consumption.

The expressions “edible product” or “edible food product” as used herein can relate to a product suitable for animal consumption and preferably a product suitable for human consumption. An edible product according to the invention can be a ready to eat (i.e finalized) food product or an intermediate in the production chain of a finalized food product. As it will be described hereafter, an edible product according to the invention can be produced in the form of a snack which may be pressed, fried and/or toasted; a sauce; a spread; a pasta; a paste; transformed meat-analogues or food specialty food such as sausage or cured sausage, pate or foie gras; a meat dough; a soup; a smoothie; a seafood; untransformed meat-analogues such as “flesh like” products.

In the following description, the term “meat” can refer to any edible part of an animal such as an animal tissue taken from a slaughtered animal. Hence, a meat can refer to liver or other offal tissues, fat tissues, muscle tissues conventionally found in an animal. The dead animal can refer to all species of the Animalia kingdom excluding human and preferably to all edible species such as a non-human vertebrate, for example, livestock, fish, bird; insect; a crustacean, for example a shrimp, prawn, crab, crayfish, and/or a lobster; a mollusk, for example an octopus, squid, cuttlefish, scallops, snail. Hence, for example, the invention allows the production of an edible product having meat-like texture such as a product mimicking foie gras, marbled beef or salmon flesh.

As used herein, the expression “in weight” is generally referring to the weight of a component compared to the weight of another component or of the whole composition, either the wet weight or the dry weight can be considered. Preferably, percentages are disclosed in reference to the wet weight.

As used herein, the expressions “cultivated cells” or “cultured cells” are used interchangeably. They can refer to cells multiplied and/or grown, preferably in a controlled environment, using a culture medium. It refers in particular to cells with a growth controlled by mankind, for example in an industrial process, as opposed to cells from conventional meat that have been multiplied in a living organism or cells grown in a natural environment (e.g. forest grown mushrooms). Cultivated cells can refer to cells belonging to Animalia kingdom for the proteins but also to Bacteria, Viridiplantae and Fungi kingdoms for example to provide additional proteins or fat. Cultivated cells can originate from cells of any origin such as cells from biopsies, from stem cells or correspond to stem cells themselves. More particularly, a cultivated cells-based protein thread can refer to a protein thread which is constituted mainly of proteins from cultivated cells. For example, a cultivated cells-based protein thread comprises at least 0.5% in weight of proteins from cultivated cells, preferably at least 1% in weight of proteins from cultivated cells, more preferably 2% in weight of proteins from cultivated cells, even more preferably 4% in weight of proteins from cultivated cells, compared to the total weight of proteins.

As used herein, the expression “extracts of cultivated cells” can refer to any fraction of disrupted cells or to any biological material purified or partially purified recovered from disrupted cells, such as cultivated cells protein extracts. Disrupted cells can be cells having partially or completely destroyed cell walls. In a protein thread, extracts of cultivated cells can comprise both disrupted cells and/or biological material recovered from disrupted cells. An extract of cultivated cells can for example be obtained by the separation and purification of the biological material recovered from disrupted cells. Hence the extracts can for example be obtained after at least a drying step, a precipitation step or solvent extraction step. An intact cultivated animal cell can refer to cultivated animal cells with an intact cell wall as it can be evaluated by microscopy.

As used herein, the expression “texturizing molecule” can refer to one or several molecules which are capable of creating a heat-resistant thread. Preferably, a heat-resistant protein thread is formed when contacting the protein matrix with the fatty matrix, said protein matrix and/or fatty matrix comprising the one or several molecules capable of creating a heat-resistant thread.

As used herein, the term “polyelectrolyte” can refer to macromolecules that, when dissolved in a polar solvent like water, have a (large) number of charged groups covalently linked to them. In general, polyelectrolytes may have various kinds of such groups. Homogeneous polyelectrolytes have only one kind of charged group, e. g. only carboxylate groups.

As used herein, the expression “heat-resistant gel” can refer to a gel, for example formed from a polyelectrolyte and a polyvalent ion, which is not liquid at a temperature below 80° C. Preferably, it refers to a gel which is not liquid at a temperature below 100° C. As used herein, the expression “heat-resistant gel-forming polyelectrolyte” can refer to a polyelectrolyte which, when combined with a suitable polyvalent ion, will form a heat-resistant gel. In particular, the heat-resistant gel-forming polyelectrolyte is combined with a polyvalent ion forming capable of complexing with the polyelectrolyte to form a heat-resistant complex. For example, the first composition comprises the polyvalent ion when the second composition comprises the polyelectrolyte, or the polyelectrolyte when said second composition comprises the polyvalent ion.

As used herein, “fatty matrix” can relate to a matrix suitable for human consumption. Preferably, a fatty matrix is constituted mainly of lipids. For example, a fatty matrix comprises at least 20% in weight of lipids, preferably at least 30% in weight of lipids, more preferably at least 40% in weight of lipids, more preferably 45% in weight of lipids, even more preferably 50% in weight of lipids.

As used herein, “protein matrix” can relate to a matrix, wet or dried, suitable for human consumption. Preferably, a protein matrix, when dried, is constituted mainly of protein. For example, a protein matrix comprises at least 50% in weight of protein, preferably at least 60% in weight of protein, more preferably 70% in weight of protein, even more preferably 80% in weight of protein with respect to a total dry weight of the protein matrix.

As used herein, “carbohydrate matrix” can relate to a matrix, wet or dried, suitable for human consumption. Preferably, a carbohydrate matrix is constituted mainly of carbohydrates. For example, a carbohydrate matrix comprises at least 50% in weight of carbohydrates, preferably at least 60% in weight of carbohydrates, more preferably 70% in weight of carbohydrates, even more preferably 80% in weight of carbohydrates with respect to the total weight of the carbohydrate matrix.

As used herein, the expressions “fermentation obtained fat” or “fermented fat” can refer to lipid molecules produced in growth reactors for example through microbial fermentation. Lipid molecules obtained by fermentation can be chemically identical to fat produced by plants or animals.

As used herein, the term “flavor” is generally the quality of the product that affects the sense of taste and/or the aroma. Hence, a “meat-like flavor” can refer to a flavor which is close to, or which approximates, the flavor of the related conventional meat product.

As used herein, the term “texture” can be considered as the “combination of the textural and structural (geometrical and surface) attributes of a food product perceptible by means of mechanical, tactile, and where appropriate, visual and auditory receptors” as defined in 2008 by the International Standards Organization (ISO, 2008, Sensory analysis—vocabular, Vols. 1-107, p. 5492). Hence, a “meat-like texture” can refer to the rheological and structural (geometrical and surface) attributes of a food product which is close to, or which approximates, the texture of the related conventional meat product (i.e. a meat product derived from animal slaughtering). An edible food product according to the invention with a meat-like texture and a meat-like flavor can be considered as an alternative to a meat product.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or even more. Hence, a composition with cells preserved substantially intact refers to a composition comprising at least about 50%, 60%, 70%, 80%, 90%, 95%, 96%, 97%, 98%, 99%, 99.5%, 99.9%, 99.99%, of intact cells.

As mentioned, the production of textured and tasty meat-like products without animal slaughtering, is a challenge for both animal welfare and the environment. In addition to animal welfare or environmental protection, it appears necessary to produce an edible food product which can correspond to the expectations of consumers by exhibiting qualities close to those of a conventional product and this with a reduced number of processing steps.

A new method has been developed for producing an edible food product comprising protein threads, which can have a meat-like texture without comprising tissues from a slaughtered animal. Moreover, said protein threads are produced embedded into the fatty matrix thus reducing the number of manufacturing steps while offering a structure that can mimic marbled meat. In particular, the answer which has been developed allows the production of an edible product comprising a fatty matrix with a texture which is greatly improved over a wide range of temperatures.

Hence, according to a first aspect, the invention relates to a method 100 of manufacturing an edible food product.

Particularly, the edible food product comprises edible protein threads embedded in a fatty matrix.

The method 100 of manufacture according to the invention will preferably allow the production of an edible food product comprising edible protein threads which can have a meat-like texture.

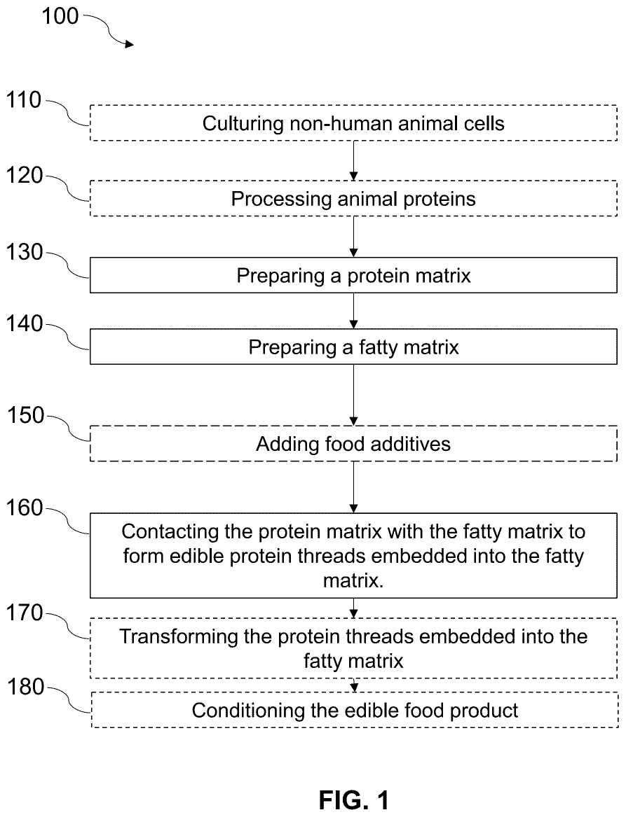

As illustrated in FIG. 1, the method comprises the steps of: preparing a protein matrix 130, preparing a fatty matrix 140, contacting 160 the protein matrix with the fatty matrix to form an edible protein thread.

An edible food product manufacturing method can comprise several other steps such as: blanching, heat sterilization, evaporation and distillation, dehydration, smoking, baking and roasting, frying, high pressure processing, Pulsed Electric Field (PEF) processing, ultrasound/cavitation/shock wave processing, pasteurization, application of cold plasma, dielectric, ohmic and infrared processing, microwave heating/blanching/assisted extraction, food irradiation, UV microbial inactivation, pulsed light technology, supercritical extractions, extrusion, freezing, chilling, modified atmospheres, drying technologies (freeze drying, membranes), fermentation, homogenization, mincing, grinding, chopping, salting, tumbling, curing, brine injection. In particular, a method according to the invention can also comprise additional steps such as: culturing 110 non-human animal cells, processing 120 animal proteins, adding food additives 150, transforming 170 the protein threads embedded into the fatty matrix, and/or conditioning 180 the edible food product.

As mentioned, an edible product can correspond to an ingredient for use in the preparation of a ready-to-eat food product such as an alternative to a meat product.

The edible food product according to the invention will preferably be a processed food product. Indeed, it will not consist in slaughtered animal flesh as such and will preferably result from a combination of edible substances from different organism sources (e.g. an hybrid product combining proteins from Animalia kingdom and fat from Plant kingdom, or combining proteins from Plant and/or Animalia and/or Fungi and/or Bacteria kingdoms and fat from Plant and/or Animalia and/or Fungi and/or Bacteria kingdoms).

As the conventional meat product, an edible food product according to the invention can comprise one or several separate matrices. At least, the edible food product according to the invention will comprise at least one protein matrix forming protein threads and at least one fatty matrix. A fatty matrix can be considered as a food matrix which contains a majority in weight of fat with respect to the total weight of the fatty matrix.

As shown in FIG. 1, a method according to the invention can comprise a step of culturing 110 non-human animal cells.

This step is in particular designed to generate an animal protein source without having to slaughter animals.

The animal cells can for example be selected from all non-human cells that can be found in usually bred, hunted or fished animals. Hence, the cultivated non-human animal cells can be selected among avian cells, bovine cells, seafood cells, porcine cells, ovine cells. Throughout the text, when animal cells are mentioned in this document, they obviously refer to non-human animal cells.

Preferably, the animal cells are selected among cells from Animalia kingdom, in particular animal cells are selected among Mammalia cells, Aves cells, Actinopterygii cells, Malacostraca cells, Mollusca cells, and combination thereof.

For example, and in a non-limiting way, Mammalia cells can be Bovidae cells, Cervidae cells, Leporidae cells or Suidae cells; Aves cells can be Anatidae cells or Phasianidae cells; Actinopterygii cells can be Gadidae cells, Merlucciidae cells, Pleuronectidae cells, Salmonidae cells, or Scombridae cells; Malacostraca cells can be Palaemonidae cells and Mollusca cells can be Cephalopoda or Bivalvia cells.

Preferably, the non-human animal cells comprise Bovidae cells, Cervidae cells, Leporidae cells, Suidae cells, Anatidae cells, Phasianidae cells, Gadidae cells, Merlucciidae cells, Pleuronectidae cells, Salmonidae cells, Scombridae cells and/or Palaemonidae cells.

More preferably, the non-human animal cells comprise Bovidae cells, Suidae cells, Anatidae cells, Phasianidae cells, Gadidae cells, Merlucciidae cells, Salmonidae cells, Scombridae cells.

Even more preferably, the non-human animal cells comprise Bovidae cells, Suidae cells, Anatidae cells, Phasianidae cells, Scombridae cells.

Whatever their origin, several types of cells can be used in this step of culturing 110.

For example, the non-human animal cells comprise cells selected among: stem cells such as embryonic stem cells, satellite cells, induced pluripotent stem cells, germ layers cells, fibro-adipogenic progenitors, muscle cells such as skeletal muscle cells, cardiac cells, or smooth muscle cells; myoblasts, myocytes, hepatocytes, fibrocytes, fibroblasts, adipocytes, chondrocytes, chondroblasts, keratinocytes, melanocytes, osteocytes, osteoblasts, Merkel cells, Langerhans cells, glial cells, Schwann cells, red blood cells (erythrocytes) and white blood cells, and combination thereof.

Preferably, the non-human animal cells comprise cells selected among: stem cells, muscle cells, fibroblasts, adipocytes, erythrocytes, and combination thereof.

There are many methods of culturing cells. These methods are often laboratory methods, but there are also many methods suitable for large volume production and human consumption. Hereafter we described some of the adapted methods for culturing non-human cells in the context of the invention.

During the step of culturing 110 non-human animal cells, cells can be either cultured in suspension or in adherence.

Advantageously, regardless of their origin, the cultivated non-human animal cells can be considered as cells grown in a culture medium. Advantageously, the culture medium does not comprise fetal bovine serum nor growth factors. The culture medium may be supplemented, preferably gradually, with hydrolysate as plant or yeast hydrolysate. Such serum free medium allows to reduce or eliminate need for animal derived components.

Various media formulations are optionally used to enable the maintenance of the capacity for self-renewal such as during expansion of the cell population. As explained, the media formulations may be modified from conventional media to not require fetal bovine serum or animal alternatives to bovine serum nor growth factors. Rather, the medium may include plant or yeast hydrolysates. Examples of plant-based formulations include soybean-based and plant hydrolysate-based media formulations. Some media formulations may further comprise at least one ingredient for enhancing the nutritional content of the cultured cells.

In addition, the medium comprises all the components and nutrients necessary to the development of cells such as salt, glucose, water, salt minerals, and amino acids.

In an embodiment, the culture medium may comprise scaffolds. The cells can be cultivated in incubators with a set up as e.g. temperature at 37° C., 5% of CO₂, at a pH of 7 and with moisture of at least 95%.

Also, the step of culturing 110 non-human animal cells can comprise a step of differentiating the cultivated cells. There are many methods of cell differentiation. These methods are often laboratory methods, but there are also many methods suitable for large volume production and human consumption. Hereafter, we described some of the adapted methods for differentiation of non-human cells in the context of the invention or using already differentiated cells. Cell differentiation may comprise production of particular proteins which contribute to the texture in a specific way more particularly related to a specific edible product.

### Differentiation of Non-Human Embryonic Stem Cells

Cells in the protein matrix may be derived from differentiation of non-human embryonic stem cells. The differentiation comprises the sum of the processes whereby undifferentiated or unspecialized cells attain their function. Stem cells can be isolated from embryos and cultured using specific cultured media in order to achieve cellular proliferation and maintenance of the undifferentiated state. This is particularly advantageous in order to reach a sufficient cell density or cell number. In an embodiment, the media formulations use synthetic serum-free media.

Next, the embryonic stem cells can be induced to differentiate for example into hepatocytes, fibroblasts, keratinocytes, myocytes or adipocytes. Differentiation can be triggered through the exposure to specific factors (e.g. growth factors or proteins) and/or culture condition (e.g. shear stress). For example, to obtain hepatocytes, non-human embryonic stem cells are induced into cells of the definitive endoderm, preferably with specific growth factors such as Activin A, WNT, FGF, or BMP; or other components having an effect on differentiation such as Insulin transferrin selenium, Rapamycin, KOSR, or Sodium butyrate. Next, cells of the final endoderm are specified into hepatic endoderm cells then hepatoblasts, preferably with specific factors as HGF, FGF, FGF and BMP. Hepatoblasts are differentiated into hepatocytes by differentiation induced with a combination of factors, preferably such as HGF, Oncostatin M, Dexamethasone and TGF-β. Next, the differentiated hepatocytes can be cultured and expanded to a desired quantity of cells.

### Differentiation of Non-Human Induced Pluripotent Stem Cells.

Cells in the protein matrix may be derived from differentiation of non-human induced pluripotent stem cells.

An episomal reprogramming strategy, for example of avian dermal fibroblasts isolated from goose, duck or chicken, can be employed to create induced pluripotent stem cells from the fibroblasts without the use of classic viral reprogramming techniques.

The induced pluripotent stem cells can be cultured using optimized media substrates and media formulations to achieve persistent cellular proliferation and maintenance of the de-differentiated state. The media formulations preferably use synthetic serum-free media. Preferably, the cells are cultured in a pathogen-free cell culture system. Next, the pluripotent stem cells can be triggered to differentiate into a specific lineage as hepatocytes and expanded to a desired quantity of cells.

### Transdifferentiated Non-Human Isolated Cells.

Cells in the protein matrix may be derived from transdifferentiated non-human isolated cells. Transdifferentiation refers to the differentiation from one differentiated cell type to another differentiated cell type, preferably, in one step. Transdifferentiation can relates to a method that modifies the differentiated phenotype or developmental potential of a cell without the formation of a pluripotent intermediate cell; i.e. it does not require that the cell be first de-differentiated (or reprogrammed) and then differentiated to another cell type. Instead, the cell type is merely “switched” from one cell type to another without going through a less differentiated phenotype. Transdifferentiation may also comprise a first step of submitting first cells having a first cell fate to conditions to generate second cells (i.e., a less differentiated cells) that are capable of differentiating into a second cell fate; and a second step of submitting the less differentiated cells to conditions triggering differentiation into cells having the second cell fate, such as hepatocyte cells.

For example, non-human cells such as embryonic fibroblasts, embryonic stem cells, satellite cells or muscle cells are isolated using techniques known in the field of cell biology as exposed above and are cultivated in a medium comprising basal medium, antibiotics, non-essential amino acids, as well as reducing agents, serum, minerals and growth factors.

### Immortalized Mature Non-Human Myocytes or Hepatocytes.

Cells in the protein matrix may be selected from immortalized mature non-human differentiated cells. Cells can be immortalized using classical techniques such as transformation or spontaneous immortalization by sequentially passing the cells until spontaneous mutation that results in immortalization. For example, in the case of hepatocytes, the specificity of the immortalized mature non-human hepatocytes lies in the fact that cells are able to divide indefinitely. Mature avian hepatocytes can be isolated from duck, goose or chicken liver. The immortalized myocytes or hepatocytes can be expanded to a desired quantity of cells and grown in culture medium.

Differentiated Cells Derived from Differentiation of Non-Human Progenitor Cells.

Cells in the protein matrix may be selected from differentiated cells derived from non-human progenitor cells. The progenitor can be grown using optimized media substrates and media formulations in order to obtain a persistent cellular proliferation and maintenance of the pluripotent state. The media formulations may comprise synthetic serum-free media. In the case of satellite cells, they are induced to differentiate into mature skeletal muscle cells and expanded to a desired quantity of cells thanks to specific and well-known differentiation factors.

As shown in FIG. 1, a method according to the invention can comprise a step of processing 120 animal proteins.

This step is in particular designed to prepare the animal proteins, especially those produced during the cell culture step, so that they are suitable for the subsequent steps.

For example, the animal proteins used in the invention can be brought in the matrix within cultivated cells that have been kept intact. Alternatively, they may be brought in the matrix with cultivated cells that have been disrupted. Also, the animal proteins can be extracted from cultivated cells, for example disrupted cultivated cells.

Hence, after the step of culturing, the cultivated cells can be preserved substantially intact or they may have been substantially disrupted for example by homogenization, extrusion, mix, blending or melt blowing, electrospinning, centrifugal spinning, blow spinning.

When the cultivated cells have been disrupted, the method according to the invention can comprise a step of extracting specific compounds after the disruption. For example, the method according to the invention can comprise a step of extracting the proteins from the cultivated cells. Proteins can be purified or can be segregated in protein fractions according to specific physicochemical properties.

As shown in FIG. 1, a method 100 of manufacturing an edible food product according to the invention comprises a step of preparing a protein matrix 130.

This step is in particular designed to define the main constituent of the protein thread and its mechanical and organoleptic properties. Hence, such a step contributes to the resolution of the problems solved by the invention.

The dry content of the protein matrix can have a marked influence on the mechanical properties of the protein threads obtained with said protein matrix. Hence, preferably the protein matrix has a dry content of at least 12 wt %. Preferably, the protein matrix has a dry content of at least 15 wt %, more preferably a dry content of at least 20 wt %. For example, the protein matrix has a dry content of at most 90 wt %. Preferably, the protein matrix has a dry content of at most 85 wt %, more preferably a dry content of at most 80 wt %.

Also, since this matrix is the basis for the constitution of protein threads, it preferably includes a significant amount of proteins. Hence, the protein matrix can comprise at least 5% in weight of proteins compared to the total wet weight of the protein matrix. Preferably, the protein matrix can comprise at least 10% in weight of proteins compared to the total wet weight of the protein matrix. Preferably, the protein matrix can comprise at least 15% in weight of proteins compared to the total wet weight of the protein matrix, more preferably at least 20% in weight, even more preferably at least 25% in weight, compared to the total wet weight of the protein matrix. The protein matrix can comprise at most 40% in weight of proteins compared to the total wet weight of the protein matrix. Preferably, the protein matrix can comprise at most 38% in weight of proteins compared to the total wet weight of the protein matrix, more preferably at most 35% in weight, even more preferably at most 30% in weight, compared to the total wet weight of the protein matrix. Hence, the protein matrix can comprise from 10% to 40% in weight of proteins compared to the total wet weight of the protein matrix. Preferably, the protein matrix can comprise from 15% to 38% in weight of proteins compared to the total wet weight of the protein matrix, more preferably from 20% to 35%, even more preferably from 25% to 30% in weight.

In the protein matrix, said proteins can be for example plant proteins, microbial proteins, algae proteins, animal proteins, fungal proteins, and a combination thereof.

In particular, the animal proteins are animal proteins from non-human cultivated animal cells. Hence, notably, the protein matrix 130 comprises animal proteins which have been obtained from non-human cultivated animal cells.

### Animal Proteins

In contrast to the conventional method of producing meat edible products, the animal proteins used here are not proteins produced from a slaughtered animal, but proteins produced by non-human animal cells grown in a cell culture facility. When animal proteins are mentioned in this document, they are obviously non-human animal proteins.

As it has been described, the animal proteins can be brought in the protein matrix in the form of intact or disrupted cultivated cells or also in the form of proteins extracted from cultivated cells.

Hence, the step of preparing a protein matrix 130 can comprise an addition of cultivated cell extracts, disrupted cultivated cells and/or intact cultivated cells. More preferably, the protein matrix comprises food-grade non-human animal cells (intact or disrupted) harvested from a cell culture (in suspension or in adherence, preferably in suspension), or extracts of said cells, preferably protein extracts of said cells.

Preferably, the protein matrix comprises cultivated non-human animal cells. The cultivated non-human animal cells can comprise disrupted cultivated cells and/or intact cultivated cells.

In particular, the protein matrix can comprise at least 1% in dry weight of cultivated non-human animal cells compared to the total wet weight of the protein matrix. Preferably, before the contacting 160 step, the protein matrix can comprise at least 2% in dry weight of cultivated non-human animal cells compared to the total wet weight of the protein matrix, more preferably at least 4% in dry weight, even more preferably at least 8% in dry weight, compared to the total wet weight of the protein matrix.

In particular, the protein matrix can comprise at most 30% in dry weight of cultivated non-human animal cells compared to the total wet weight of the protein matrix. Preferably, before the contacting 160 step, the protein matrix can comprise at most 28% in dry weight of cultivated non-human animal cells compared to the total wet weight of the protein matrix, more preferably at most 25% in dry weight, even more preferably at most 22% in dry weight, compared to the total wet weight of the protein matrix.

In particular, the protein matrix can comprise cultivated non-human animal cells from 1% to 30% in dry weight compared to the total wet weight of the protein matrix. Preferably, the protein matrix can comprise cultivated non-human animal cells from 2% to 28% in dry weight compared to the total wet weight of the protein matrix, more preferably from 4% to 25% in dry weight, even more preferably from 8% to 22% in dry weight, compared to the total wet weight of the protein matrix.

The quantity of cultivated non-human animal cells can be measured after harvesting and centrifugation and corrected based on the moisture content. The moisture content can be measured according to the international norm ISO 1442:1997. When implementing a method of producing an edible product or ingredient, the origin of the ingredient is generally known. Hence, the person skilled in the art will be able to know if he is adding proteins that are either animal proteins being from non-human cultivated animal cells or not. An animal protein being from non-human cultivated animal cells is for example a protein produced by a non-human animal cell which is grown in a bioreactor or fermentation reactor.

In particular, the protein matrix can comprise at least 1% in dry weight of cultivated non-human animal cell extracts compared to the total wet weight of the protein matrix. Preferably, before the contacting 160 step, the protein matrix can comprise at least 2% in dry weight of cultivated non-human animal cell extracts compared to the total wet weight of the protein matrix, more preferably at least 4% in dry weight, even more preferably at least 8% in dry weight, compared to the total wet weight of the protein matrix.

In particular, the protein matrix can comprise at most 30% in dry weight of cultivated non-human animal cell extracts compared to the total wet weight of the protein matrix. Preferably, before the contacting 160 step, the protein matrix can comprise at most 28% in dry weight of cultivated non-human animal cell extracts compared to the total wet weight of the protein matrix, more preferably at most 25% in dry weight, even more preferably at most 22% in dry weight, compared to the total wet weight of the protein matrix.

In particular, the protein matrix can comprise cultivated non-human animal cell extracts from 1% to 30% in dry weight compared to the total wet weight of the protein matrix. Preferably, before the contacting 160 step, the protein matrix can comprise cultivated non-human animal cell extracts in an amount that is from 2% to 28% in dry weight compared to the total wet weight of the protein matrix, more preferably from 4% to 25% in dry weight, even more preferably from 8% to 22% in dry weight, compared to the total wet weight of the protein matrix.

The concentration of proteins, and in particular animal proteins, in the protein matrix can affect the texture of the protein thread.

The protein matrix can comprise at least 0.5% in weight of animal proteins being from non-human cultivated animal cells compared to the total wet weight of the protein matrix. Preferably, the protein matrix can comprise at least 1% in weight of animal proteins being from non-human cultivated animal cells compared to the total wet weight of the protein matrix, more preferably at least 2% in weight, even more preferably at least 4% in weight, compared to the total wet weight of the protein matrix.

The protein matrix can comprise at most 30% in weight of animal proteins being from non-human cultivated animal cells compared to the total wet weight of the protein matrix. Preferably, the protein matrix can comprise at most 28% in weight of animal proteins being from non-human cultivated animal cells compared to the total wet weight of the protein matrix, more preferably at most 25% in weight, even more preferably at most 22% in weight, compared to the total wet weight of the protein matrix.

Hence, the protein matrix can comprise from 0.5% to 30% in weight of animal proteins being from non-human cultivated animal cells compared to the total wet weight of the protein matrix. Preferably, the protein matrix can comprise from 1% to 28% in weight of animal proteins being from non-human cultivated animal cells compared to the total wet weight of the protein matrix, more preferably from 2% to 25%, even more preferably from 4% to 22% in weight, compared to the total wet weight of the protein matrix.

The protein matrix can comprise at least 5% in weight of animal proteins being from non-human cultivated animal cells compared to the total weight of proteins in the protein matrix. Preferably, the protein matrix can comprise at least 10% in weight of animal proteins being from non-human cultivated animal cells compared to the total weight of proteins in the protein matrix, more preferably at least 20% in weight, even more preferably at least 30% in weight, at least 40% in weight, at least 50% in weight, at least 60% in weight, compared to the total weight of proteins in the protein matrix.

The protein matrix can comprise at most 100% in weight of animal proteins being from non-human cultivated animal cells compared to the total weight of proteins in the protein matrix. Preferably, the protein matrix can comprise at most 90% in weight of animal proteins being from non-human cultivated animal cells compared to the total weight of proteins in the protein matrix, more preferably at most 80% in weigh compared to the total weight of proteins in the protein matrix.

Hence, the protein matrix can comprise from 5% to 100% in weight of animal proteins being from non-human cultivated animal cells compared to the total weight of proteins in the protein matrix. Preferably, the protein matrix can comprise from 10% to 100% in weight of animal proteins being from non-human cultivated animal cells compared to the total weight of proteins in the protein matrix, more preferably from 20% to 100%, even more preferably from 30% to 100% in weight, from 40% to 90% in weight, from 50% to 100% in weight, from 50% to 90% in weight, compared to the total weight of proteins in the protein matrix.

Preferably, the quantity of animal proteins can be measured using Kjeldahl titration according to the international norm ISO 937:1978. When implementing a method of producing an edible product or ingredient, the origin of the ingredient is generally known. Hence, the person skilled in the art implementing the invention will be able to know if he is adding proteins that are either animal proteins being from non-human cultivated animal cells or not.

In the context of the present invention, the presence of specific molecules, in particular proteins, can be of utmost importance to improve the final protein thread quality. The quantity of specific molecules, in particular proteins, can be measured by well-known techniques by the person skilled in the art such as enzyme-linked immunosorbent assay or mass spectrometry. For example, the animal proteins can comprise heat shock proteins.

Preferably, the animal proteins comprise myofibrillar proteins. For example, the myofibrillar proteins can comprise actin, myosin, tropomyosin, troponin, actinin, connectin, titin, nebulin, C protein, M proteins, desmin or a combination thereof.

Preferably, the myofibrillar proteins are selected among: actin, desmin, myosin, troponin, titin, nebulin or a combination thereof. More preferably, the myofibrillar proteins are selected among: actin, desmin, myosin, troponin or a combination thereof. Even more preferably, the myofibrillar proteins are selected among: actin, myosin or a combination thereof.

The protein matrix can comprise at least 0.25% in weight of myofibrillar proteins compared to the total weight of proteins, preferably at least 0.5% in weight of myofibrillar proteins, more preferably at least 1% in weight of myofibrillar proteins, even more preferably at least 2% in weight of myofibrillar proteins compared to the total weight of proteins in the protein matrix.

The protein matrix can comprise at most 70% in weight of myofibrillar proteins compared to the total weight of proteins, preferably at most 60% in weight of myofibrillar proteins, more preferably at most 50% in weight of myofibrillar proteins, even more preferably at most 40% in weight of myofibrillar proteins compared to the total weight of proteins in the protein matrix.

Hence, the protein matrix can comprise between 0.25% and 70% in weight of myofibrillar proteins compared to the total weight of proteins, preferably between 0.5% and 60% in weight of myofibrillar proteins, more preferably between 1% and 50% in weight of myofibrillar proteins, even more preferably between 2% and 40% in weight of myofibrillar proteins compared to the total weight of proteins in the protein matrix.

In particular, the protein matrix can advantageously comprise specific myofibrillar proteins. For example, the protein matrix can comprise troponin protein preferably at least 0.0125% in weight of troponin protein compared to the total weight of proteins in the protein matrix, preferably at least 0.025% in weight, more preferably at least 0.05% in weight, even more preferably at least 0.1% in weight, compared to the total weight of proteins in the protein matrix.

The protein matrix can comprise at most 3.5% in weight of troponin protein compared to the total weight of proteins in the protein matrix, preferably at most 3% in weight, more preferably at most 2.5% in weight, even more preferably at most 2% in weight, compared to the total weight of proteins in the protein matrix.

Hence, the protein matrix can comprise from 0.0125 to 3.5% in weight of troponin protein compared to the total weight of proteins in the protein matrix, preferably from 0.025 to 3% in weight, more preferably from 0.05 to 2.5% in weight; even more preferably from 0.1 to 2% in weight, compared to the total weight of proteins in the protein matrix.

The troponin can be produced by a cultivated animal cell. However, it can also be a recombinant troponin that can be produced by a cultivated cell which can be a plant cell, an insect cell or a microbial cell.

In the context of the present invention, the presence of some specific molecules can improve the produced protein thread. In particular, the presence of extracellular matrix animal molecules (ECM) such as ECM polysaccharides, ECM proteoglycans or ECM proteins, can improve the produced protein thread. ECM proteins are particularly preferred.

Such molecules can be added in the protein matrix through cultivated cells, preferably cultivated animal cells, such as fibroblasts, known to produce such molecules.

The protein matrix can comprise collagen, preferably at least 1% in weight of collagen compared to the total weight of proteins in the protein matrix, preferably at least 2% in weight, more preferably at least 3% in weight, even more preferably at least 4% in weight, compared to the total weight of proteins in the protein matrix.

The protein matrix can comprise at most 16% in weight of collagen compared to the total weight of proteins in the protein matrix, preferably at most 14%, more preferably at most 12%, even more preferably at most 10% in weight, compared to the total weight of proteins in the protein matrix.

Hence, the protein matrix can comprise from 1 to 16% in weight of collagen compared to the total weight of proteins in the protein matrix, preferably from 2 to 14% in weight, more preferably from 3 to 12% in weight; even more preferably from 4 to 10% in weight, compared to the total weight of proteins in the protein matrix.

The collagen can be a collagen produced by a cultivated animal cell. However, it can also be a recombinant collagen that can be produced by a cultivated cell which can be a plant cell, an insect cell or a microbial cell.

The protein matrix can comprise elastin and/or tropoelastin, preferably at least 0.065% in weight of elastin and/or tropoelastin compared to the total weight of proteins in the protein matrix, preferably at least 0.13% in weight, more preferably at least 0.2% in weight, even more preferably at least 0.25% in weight, compared to the total weight of proteins in the protein matrix.

The protein matrix can comprise at most 7.5% in weight of elastin and/or tropoelastin compared to the total weight of proteins in the protein matrix, preferably at most 7% in weight, more preferably at most 6.5% in weight, even more preferably at most 6% in weight, compared to the total weight of proteins in the protein matrix.

Hence, the protein matrix can comprise from 0.065 to 7.5% in weight of elastin and/or tropoelastin compared to the total weight of proteins in the protein matrix, preferably from 0.13 to 7% in weight, more preferably from 0.2 to 6.5% in weight; even more preferably from 0.25 to 6% in weight, compared to the total weight of proteins in the protein matrix.

Preferably, said elastin and/or tropoelastin are produced by a cultivated animal cell. However, it can also be a recombinant elastin and/or tropoelastin that can be produced by a cultivated cell which can be a plant cell, an insect cell or a microbial cell.

As it has been described, in a preferred embodiment the protein matrix comprises animal proteins being from non-human cultivated animal cells. However, the protein matrix can comprise proteins from other origins.

In particular, the step of preparation of a protein matrix 130 can comprise the addition of non-animal proteins such as plant proteins, microbial proteins, algae proteins and/or fungal proteins. As it is mentioned in the examples, adding some non-animal proteins can be used to tune the viscoelasticity or flow properties of the protein matrix comprising non-human animal cultivated cells.

In a particular embodiment, the protein matrix further comprises plant proteins, preferably the plant proteins can be selected among: sunflower proteins, soybeans proteins, pea proteins, canola proteins, mung bean proteins, chickpea proteins, Faba bean proteins, lentils proteins, seaweed proteins, potato proteins, Quinoa proteins, nuts proteins, wheat proteins, winged bean proteins, bambara bean proteins, dulse proteins, mesquite bean proteins, duckweed proteins, dry broad bean proteins, dry cow pea proteins, lupins proteins, jackfruit proteins, amaranth proteins, millet proteins, oat proteins, chia proteins, hemp seed proteins and rice proteins or a combination thereof.

Preferably, the step of preparation of a protein matrix 130 can comprise the addition of non-animal proteins from at least two different sources. For example, the protein matrix comprises plant proteins from at least two different plants. Indeed, a combination of several origins allows an improvement of the properties of the edible protein threads and the establishment of an amino acid profile optimized for human nutrition. More preferably the step of preparation of a protein matrix 130 can comprise the addition of pulse proteins and cereal proteins.

As shown in FIG. 1, a method 100 of manufacturing an edible food product according to the invention comprises a step of preparing a fatty matrix 140.

This step is in particular designed to define one of the main constituents of the edible food product, in particular its mechanical and organoleptic properties. Hence, such a step contributes to the resolution of the problems solved by the invention. Moreover, as it will be illustrated in the examples, the content of this fatty matrix has a marked influence on the experience of the product taster. Also, this step is particularly designed to prepare a fatty matrix that is complementary to the protein matrix to form a protein thread with the expected texture when contacting the two matrices. Hence, such a step contributes to the resolution of the problems solved by the invention.

The fatty matrix preparation can comprise the addition of fat which is, at 20° C., in a solid state, a liquid state and/or a semi-solid state. Preferably, the fatty matrix preparation comprises the addition of fat which is in a solid state at 20° C. In an embodiment, the fatty matrix preparation comprises the addition of at least 10% of fat which is in a solid state at 20° C.

While contacting the protein matrix with the fatty matrix, the fatty matrix should have an appropriate viscosity in order to allow the formation of protein threads into the fatty matrix. The fat used to prepare the fatty matrix should be melted if they are solid or semi-solid at 20° C.

Hence, the preparation step of the fatty matrix can comprise a heat treatment of the fat to be used in the fatty matrix. Preferably, the fat used in the fatty matrix has been heated at a temperature of at least 50° C. All the fat to be used in the fatty matrix can be heated or only a portion. This is particularly relevant when at least a portion of the fat used is not liquid at 20° C.

As mentioned, the fatty matrix comprises at least 20.00% in weight of fat, in particular at least 20.00% in weight of plant fat and/or fermented fat, compared to the total weight of the fatty matrix. For example, the fatty matrix comprises at least 30.00% in weight of fat, in particular at least 30.00% in weight of plant fat and/or fermented fat, compared to the total weight of the fatty matrix.

In particular, the fatty matrix comprises more than 40.00% of plant fat and/or fermented fat in weight with respect to a total wet weight of fatty matrix. For example, the fatty matrix can comprise at least 50% of plant fat and/or fermented fat in weight with respect to a total weight of fatty matrix. Preferably, the fatty matrix comprises at least 55%, more preferably at least 60%, even more preferably at least 65%, such as at least 70% of plant fat and/or fermented fat in weight with respect to a total weight of fatty matrix (e.g. wet weight). It should be understood that when the fat matrix contains plant fat and fermented fat, then the percentages mentioned above refer to the combined percentages in weight of plant and fermented fat. Preferably said fat is a plant fat or a mixture of fat from different plants.

However, as will be specified later, the fatty phase does not only include plant fat and/or fermented fat. It can include fat of other origins and ingredients other than fat. Hence, the fatty matrix can comprise at most 95% of plant fat and/or fermented fat in weight with respect to a total weight of fatty matrix. Preferably, the fatty matrix comprises at most 90%, more preferably at most 85%, even more preferably at most 80%, of plant fat and/or fermented fat in weight with respect to a total weight of fatty matrix (e.g. wet weight).

The fatty matrix according to the invention can be characterized by its ability to adequately mimic the properties of the conventional fat from meat. In particular, as it will be mentioned in the example section, the fatty matrix can have a hardness at cooking temperature which is substantially equal to the hardness of the lipid phase of conventional meat at this cooking temperature.

Preferably, the fatty matrix comprises a liquid fat and/or a solid fat. Preferably, the fatty matrix comprises a combination of liquid fat and a solid fat, at 20° C.

The edible food product may have better meat similarity properties depending on the amount of saturated and unsaturated fatty acids. For example, the fatty matrix comprises a ratio in weight of unsaturated C18 fatty acids on saturated C16-C18 fatty acids of at least 0.80, preferably at least 1.00, more preferably at least 1.20, even more preferably at least 1.50. For example, the fatty matrix comprises a ratio in weight of unsaturated C18 fatty acids on saturated C16-C18 fatty acids ranging from 0.8 to 5, more preferably ranging from 0.8 to 4, more preferably ranging from 1.00 to 3, even more preferably a ratio in weight between unsaturated C18 fatty acids and saturated C16-C18 fatty acids ranging from 1.50 to 2.50.

Unsaturated fatty acids can be selected among monounsaturated fatty acids and polyunsaturated fatty acids. Hence, unsaturated C18 fatty acids with less than four carbon-carbon double bonds can be selected among oleic acid, linoleic acid, linolenic acid, and a combination thereof. Polyunsaturated C18 fatty acids can be selected among linoleic acid, linolenic acid and a combination thereof. It should be understood that when unsaturated fatty acid amounts are mentioned, they include monounsaturated fatty acid amounts and polyunsaturated fatty acids.

Preferably, the unsaturated fatty acids and saturated fatty acids in the fatty matrix can be measured using gas chromatography coupled with a flame ionization detector. The fatty acids are typically converted to fatty acid methyl esters (FAMEs) which are more easily separated and thus quantified than their original form within triglycerides or as free fatty acids. In particular, it can be measured following the instructions detailed in the norm ISO 12966.

Moreover, the fatty acids can be free or present in the form of glycerides, such as monoglycerides, diglycerides or triglycerides. As it will be detailed hereafter, the fatty acids are preferably mainly present in the form of triglycerides. As it will be described, the plant fat and/or fermented fat comprise triglycerides. Moreover, fatty acids, fatty acid composition and in particular fatty acids ratio described above can refer to fatty acids in the form of triglycerides.

Preferably, most of the plant fat and/or fermented fat found in the fatty matrix are present in the form of triglycerides. Hence, the fatty matrix can comprise at least 50% in weight of triglycerides with respect to a total weight of fat in the fatty matrix, preferably at least 70% in weight of triglycerides with respect to a total weight of fat in the fatty matrix, more preferably at least 80% in weight of triglycerides with respect to a total weight of fat in the fatty matrix, even more preferably at least 90% such as at least 95% in weight of fats are triglycerides with respect to a total weight of fat in the fatty matrix. In an embodiment, these triglycerides can come from the plant and/or fermented fat but also from cultivated cells added to the fatty matrix.

In particular, and as it will be shown in examples, the relative quantities of unsaturated C18 fatty acids such oleic acid, linoleic acid, linolenic acid; and saturated C16-C18 fatty acids such as stearic acid and palmitic acid in the fatty matrix are important to obtain a texture as close as possible to the texture of a meat product.

A way of solving the problem is when a high portion of unsaturated C18 fatty acids (e.g. with less than four carbon-carbon double bonds), preferably in the form of triglycerides, is added in the fatty matrix. Hence, in particular, as it will be shown in examples, the triglycerides of the fatty matrix can comprise more than 40% in weight of unsaturated C18 fatty acids with less than four carbon-carbon double bonds with respect to the total weight of triglycerides in the fatty matrix. Preferably, the triglycerides of the fatty matrix comprise more than 45% in weight of unsaturated C18 fatty acids with less than four carbon-carbon double bonds with respect to the total weight of triglycerides in the fatty matrix. More preferably, the triglycerides of the fatty matrix comprise more than 50% in weight of unsaturated C18 fatty acids with less than four carbon-carbon double bonds with respect to the total weight of triglycerides of the fatty matrix. Even more preferably, the triglycerides comprise more than 55% in weight of unsaturated C18 fatty acids with less than four carbon-carbon double bonds with respect to the total weight of triglycerides in the fatty matrix.

As detailed in the examples, the edible food product of the invention can also comprise a minimal concentration of polyunsaturated C18 fatty acids, preferably in the form of triglycerides. Hence, in particular, as it will be shown in examples, the triglycerides of the fatty matrix comprise more than 1.5% in weight of polyunsaturated C18 fatty acids with respect to the total weight of triglycerides in the fatty matrix. Preferably, the triglycerides of the fatty matrix comprise more than 2% in weight of polyunsaturated C18 fatty acids with respect to the total weight of triglycerides in the fatty matrix. Preferably, the triglycerides of the fatty matrix comprise more than 2.5% in weight of polyunsaturated C18 fatty acids with respect to the total weight of triglycerides in the fatty matrix. More preferably, the triglycerides of the fatty matrix comprise more than 3.5% in weight of polyunsaturated C18 fatty acids with respect to the total weight of triglycerides of the fatty matrix. Even more preferably, the triglycerides comprise at least 7% in weight of polyunsaturated C18 fatty acids with respect to the total weight of triglycerides in the fatty matrix. In particular, the edible product of the invention preferably comprises a minimal concentration of linolenic acids, preferably in the form of triglycerides. Hence, in particular, as it will be shown in examples, the triglycerides of the fatty matrix comprise linolenic acids for example more than 0.01% in weight of linolenic acids with respect to the total weight of triglycerides in the fatty matrix. Preferably, the triglycerides of the fatty matrix comprise more than 0.10% in weight of linolenic acids with respect to the total weight of triglycerides in the fatty matrix. More preferably, the triglycerides of the fatty matrix comprise at least 1% in weight of linolenic acids with respect to the total weight of triglycerides of the fatty matrix. Even more preferably, the triglycerides comprise at least 2% in weight of linolenic acids with respect to the total weight of triglycerides in the fatty matrix.

Similarly, it has been found that presence of stearic acid in the triglycerides can be beneficial to the needs solved by the invention. Hence, in particular, as it is illustrated in examples, the triglycerides of the fatty matrix can comprise at least 4% in weight of stearic acid with respect to the total weight of triglycerides in the fatty matrix. Preferably, the triglycerides of the fatty matrix comprise at least 5% in weight of stearic acid with respect to the total weight of triglycerides in the fatty matrix. More preferably, the triglycerides of the fatty matrix comprise at least 10% in weight of stearic acid with respect to the total weight of triglycerides of the fatty matrix. Even more preferably, the triglycerides comprise at least 15% in weight of stearic acid with respect to the total weight of triglycerides in the fatty matrix. However, an excessive portion of stearic acid in the triglycerides of the fatty matrix can be detrimental to the needs solved by the invention. In preferred embodiments, the triglycerides of the fatty matrix comprise less than 40% in weight of stearic acid with respect to the total weight of triglycerides in the fatty matrix. Preferably, the triglycerides of the fatty matrix comprise more than 30% in weight of stearic acid with respect to the total weight of triglycerides in the fatty matrix. More preferably, the triglycerides of the fatty matrix comprise more than 20% in weight of stearic acid with respect to the total weight of triglycerides of the fatty matrix.

As it is illustrated in examples, an excessive amount of saturated C16-C18 fatty acid with respect to a total weight of plant and/or fermented fat triglycerides can be detrimental to the needs solved by the invention. Hence, for example, the fatty matrix comprises less than 60% in weight of saturated C16-C18 fatty acid with respect to a total weight of plant and/or fermented fat triglycerides; preferably less than 50% in weight of saturated C16-C18 fatty acid with respect to a total weight of plant and/or fermented fat triglycerides; more preferably less than 40% in weight of saturated C16-C18 fatty acid with respect to a total weight of plant and/or fermented fat triglycerides; even more preferably less than 30% in weight of saturated C16-C18 fatty acid with respect to a total weight of plant and/or fermented fat triglycerides.

It has been discovered that an excessive portion of palmitic acid in the triglycerides of the fatty matrix can be detrimental to the needs solved by the invention. Hence, in particular, as it is shown in examples, the triglycerides of the fatty matrix comprise less than 59.00% in weight of palmitic acid with respect to the total weight of triglycerides in the fatty matrix. Preferably, the triglycerides of the fatty matrix comprise less than 50% in weight of palmitic acid with respect to the total weight of triglycerides in the fatty matrix. More preferably, the triglycerides of the fatty matrix comprise less than 40% in weight of palmitic acid with respect to the total weight of triglycerides in the fatty matrix. Even more preferably, the triglycerides of the fatty matrix comprise at most 35.00% in weight of palmitic acid with respect to the total weight of triglycerides in the fatty matrix.

On the contrary, a high proportion of oleic acid in the triglycerides is found beneficial to the needs solved by the invention. Hence, in particular, as it will be shown in examples, the triglycerides of the fatty matrix comprise more than 33.00% in weight of oleic acid with respect to the total weight of triglycerides in the fatty matrix. Preferably, the triglycerides of the fatty matrix comprise more than 35% in weight of oleic acid with respect to the total weight of triglycerides in the fatty matrix. More preferably, the triglycerides of the fatty matrix comprise more than 40% in weight of oleic acid with respect to the total weight of triglycerides of the fatty matrix. Even more preferably, the triglycerides comprise more than 45% in weight of oleic acid with respect to the total weight of triglycerides in the fatty matrix.

As mentioned, the weight of total oleic acid, palmitic acid and stearic acid in the fatty matrix (free, as well as contained in monoglycerides, diglycerides and triglycerides) can be measured by converting the free fatty acids monoglycerides, diglycerides and triglycerides into fatty acid methyl esters and to quantify them using gas chromatography coupled with flame ionization detector or with mass spectrometer. For example, this can be done through the norm ISO 12966. Hence, the person skilled in the art can easily calculate the portion in weight of oleic acid, palmitic acid and stearic acid in the triglycerides.

In particular, as it is illustrated in examples, the triglycerides in the fatty matrix can comprise:
less than 50% in weight of palmitic acid with respect to the total weight of the fatty matrix triglycerides,
more than 35% in weight of oleic acid with respect to the total weight of the fatty matrix triglycerides, and
at least 2% in weight of linoleic acid with respect to the total weight of the fatty matrix triglycerides.

Preferably, as it is illustrated in examples, the triglycerides in the fatty matrix comprise:
less than 50% in weight of palmitic acid with respect to the total weight of the fatty matrix triglycerides,
at least 4% in weight of stearic acid with respect to the total weight of the fatty matrix triglycerides,
more than 35% in weight of oleic acid with respect to the total weight of the fatty matrix triglycerides, and
at least 2% in weight of linoleic acid with respect to the total weight of the fatty matrix triglycerides.

More preferably, as it is illustrated in examples, the triglycerides in the fatty matrix comprise:
less than 50% in weight of palmitic acid with respect to the total weight of the fatty matrix triglycerides,
at least 4% in weight of stearic acid with respect to the total weight of the fatty matrix triglycerides,
more than 35% in weight of oleic acid with respect to the total weight of the fatty matrix triglycerides,
more than 5% in weight of linoleic acid with respect to the total weight of the fatty matrix triglycerides, and
more than 0.01% in weight of linolenic acid with respect to the total weight of the fatty matrix triglycerides.

The fatty matrix can only comprise plant fat or can only comprise fermented fat or can comprise a combination of plant fat and fermented fat. Moreover, the plant fat and/or fermented fat can be completed with other fats. Indeed, as the fatty matrix can comprise proteins such as animal proteins which can come from animal cultured cells, the fatty matrix can comprise animal cell triglycerides. Preferably the fats of the fatty matrix are mainly selected from plant fat and/or fermented fat. Also, the fats of the fatty matrix can be obtained from fractionated oil and/or hydrogenated oil and/or deodorized oil and/or interesterified oil.

The plant fat can for example comprise fat or oil extracted from edible plant matter, including the flowers, fruits, stems, leaves, roots, germs and seeds. Preferably, the plant fat can for example comprise fat or oil extracted from oilseeds or fruits. In particular, the plant fat can relate to fat extracted from canola seed (rapeseed), castor, coconut, flaxseed, allanblackia, olive, sunflower, soybean, peanut, illipe, cottonseed, shea, palm, avocado, safflower, sesame, lemon, grapeseed, macadamia, almond, sal, kokum, or mango or a combination thereof.

In particular, the plant fat used according to the invention can be selected from olive oil, palm oil, avocado oil, almond oil or combination thereof.

The fermented fat can comprise fat or oil extracted from cells cultivated in anaerobic or aerobic fermentation process, in particular process involving cultivation of oleaginous microorganisms or animal cells excluding human cells. For example, the fermented fat can include fat from cyanobacteria, microalgae, yeast, fungi such as filamentous fungi, bacteria or cultivated animal cells excluding human cells, such as adipocytes. Preferably, the fermented fat comprises fat or oil extracted from oleaginous yeast, such as Rhodosporidium toruloides, Lipomyces starkeyi and Yarrowia lipolytica.

Moreover, beside the presence of fat, the fatty matrix according to the invention advantageously can comprise proteins. Said proteins of the fatty matrix could be non-human animal proteins, plant proteins, microbial proteins, algae proteins and/or fungal proteins, and a combination thereof.

In a particular embodiment, proteins are animal proteins excluding human proteins also called non-human animal proteins. Indeed, without being limited by the theory, the presence of proteins, in particular animal proteins, improves the ability of the fatty matrix to respond to the identified needs. For example, the inventors formulated the hypothesis that, in the specific fatty matrix of the invention, the proteins help in combination with a specific fat composition to produce a suitable mouthfeel, similar to the meat texture and in particular the fat phase texture of the meat. Moreover the presence of proteins in the fatty matrix contributes to limiting the additives present in the food matrix. Moreover, the presence of animal proteins, preferably from non-human cultivated animal cells or extracts of said cells, improves the organoleptic properties of the fatty matrix, especially the flavor.

Hence, as it is illustrated in examples, the fatty matrix can further comprise at least 0.25% of proteins in weight with respect to the total weight of fatty matrix (e.g. wet weight). Preferably, the fatty matrix further comprises at least 0.50% of proteins in weight with respect to the total weight of the fatty matrix. More preferably, the fatty matrix further comprises at least 1% of proteins in weight with respect to the total weight of the fatty matrix. Even more preferably, the fatty matrix further comprises at least 1.5% of proteins in weight with respect to the total weight of fatty matrix (e.g. wet weight). These proteins can be plant proteins, fungal proteins, bacterial proteins, fermented proteins, recombinant proteins, non-human animal proteins and a mixture thereof. Advantageously said proteins include non-human animal proteins. In an even preferred embodiment, said non-human animal proteins being from non-human cultivated animal cells or extracts of said cultivated cells. Thus, at least 0.25% of proteins in weight with respect to the total weight of fatty matrix can be non-human animal proteins, preferably from non-human cultivated animal cells or extracts of said cells, advantageously improving the flavor of the edible food product according to the invention.

Hence, as it is illustrated in examples, the fatty matrix can further comprise at least 0.25% in weight of non-human animal proteins with respect to the total wet weight of the fatty matrix, preferably at least 0.5% of non-human animal proteins in weight with respect to the total weight of the fatty matrix (e.g. wet weight). More preferably, the fatty matrix further comprises at least 1.0% of non-human animal proteins in weight with respect to the total weight of the fatty matrix. Even more preferably, the fatty matrix further comprises at least 1.5% of non-human animal proteins in weight with respect to the total weight of the fatty matrix (e.g. wet weight). Preferably, said non-human animal proteins being from non-human cultivated animal cells or extracts of said cells.

However, the fatty matrix preferably should not comprise a high quantity of proteins and in particular non-human animal proteins. Hence, the fatty matrix can comprise less than 20% of proteins in weight with respect to the total weight of the fatty matrix. Preferably, the fatty matrix comprises less than 18% of proteins in weight with respect to the total weight of the fatty matrix. More preferably, the fatty matrix further comprises less than 16% of proteins in weight with respect to the total weight of the fatty matrix. Even more preferably, the fatty matrix further comprises less than 14% of proteins in weight with respect to the total weight of the fatty matrix (e.g. wet weight). Among these proteins, the fatty matrix can comprise less than 16%, less than 14%, less than 12%, less than 10% of non-human animal proteins in weight with respect to the total weight of the fatty matrix (e.g. wet weight). Preferably, said non-human animal proteins being from non-human cultivated animal cells or extracts of said cells.

Preferably, the fatty matrix further comprises from 0.25% to 20% in weight of proteins with respect to the total weight of fatty matrix. More preferably, the fatty matrix further comprises from 0.5% to 18% in weight of proteins with respect to the total weight of fatty matrix. Even more preferably, the fatty matrix further comprises from 1% to 16% in weight of proteins with respect to the total weight of fatty matrix (e.g. wet weight). Among these proteins, the fatty matrix can comprise from 0.25% to 16%, from 0.5% to 14%, from 1% to 12%, or from 1.5% to 10%, of non-human animal proteins in weight with respect to the total weight of the fatty matrix. Preferably, said non-human animal proteins being from non-human cultivated animal cells or extracts of said cells.

Non-human animal cells, non-human animal proteins and animal proteins from cultivated non-human animal cells and extracts of said cells have already been described, for example when describing the step of culturing 110 non-human animal cells. Hence, all embodiments related to non-human animal cells, non-human animal proteins and animal proteins from cultivated non-human animal cells and extracts of said cells described here before, preferred or not preferred, are applicable to the non-human animal protein that can be found in the fatty matrix. In particular non-human animal protein can be selected among Bovidae proteins, Aves proteins, Suidae proteins, Leporidae proteins, Actinopterygii proteins, and combination thereof. More preferably, the non-human animal proteins are selected among duck proteins, goose proteins, chicken proteins, beef proteins, pork proteins, tuna proteins, salmon proteins and combination thereof.

In an embodiment, the animal proteins of the fatty matrix can be derived from cultured animal cells, excluding human cells. For those who do not understand this meaning, the person skilled in the art knows that a beef protein is a protein produced by a beef cell, said beef cell may be cultured outside the beef organism. For example, a beef cell belongs or is from an organism which belongs, by its ancestors, to a kingdom (Animalia), a phylum (Chordata), a class (Mammalia), an order (Artiodactyla), a family (Bovidae) and to a genus (Bos). For example it can belong to species such as Bos taurus.

### Texturizing Molecule Capable of Creating a Heat-Resistant Thread

As it has been mentioned, the protein matrix and/or the fatty matrix also comprise one or more texturizing molecules.

The one or more texturizing molecules are capable of creating a heat-resistant thread. Preferably, a heat-resistant protein thread is formed when contacting the protein matrix with the fatty matrix.

The texturizing molecule can comprise molecule(s) capable of creating ionic bonds or covalent bonds involving said proteins, preferably non-human animal proteins.

For example, the texturizing molecule can comprise an enzyme which can create covalent bonds involving proteins, preferably bonds between proteins.

The protein matrix and/or the fatty matrix, can further comprise a cross-linking molecule such as peptidase, α-galactosidase, alcalase, thermolysin, pepsin, tripsin, chymotrypsin, asparaginase, elastase, subtilisin, glucose oxidase, laccase, transglutaminase, pectin esterase, sortase, tyrosinase, oxidoreductase such as lysyl oxidase and peroxidase, genipin, riboflavin, mono amine oxidase or a combination thereof, preferably further comprises laccase, transglutaminase or a combination thereof. Preferably, the texturizing molecule comprises transglutaminase.

Such molecules, having cross-linking activity, can act as the texturizing molecule in the protein matrix and/or in the fatty matrix.

One or more texturizing molecules can be present in the protein matrix and/or in the fatty matrix.

For example, one or more texturizing molecules can comprise a hydrocolloid found in the protein matrix.

Preferably, the hydrocolloid can be selected among: methyl-cellulose, kappa-carrageenan, iota-carrageenan, lambda-carrageenan, pullulan, xanthan, konjac, dextran, starch, gelatin, gellan gum low acyl, gellan gum high acyl, gluten, agar, guar gum, gum arabic, locust bean gum or combination thereof.

More preferably, the hydrocolloid can be selected among: methyl-cellulose, lambda-carrageenan, pullulan, xanthan, konjac, gellan gum low acyl, gellan gum high acyl, or combination thereof.

In an embodiment, one or more texturizing molecules are present in the protein matrix and in the fatty matrix.

In particular, one or more texturizing molecules comprise molecules that form a heat-resistant gel when combined. Preferably, the one or more texturizing molecules comprise a polyelectrolyte and a polyvalent ion forming a heat-resistant gel when combined. For example, the polyelectrolyte is capable of complexing with the polyvalent ion to form a heat-resistant complex.

In that case, the fatty matrix 140 comprises the polyvalent ion when the protein matrix 130 preparation step comprises an addition of the polyelectrolyte, or the polyelectrolyte when said protein matrix 130 preparation step comprises an addition of the polyvalent ion. Hence, in particular, the step of preparing a fatty matrix 140 comprises adding in the fatty matrix the polyvalent ion when the protein matrix 130 preparation step comprises an addition of the polyelectrolyte, or the polyelectrolyte when said protein matrix 130 preparation step comprises an addition of the polyvalent ion.

It should be noted that when the protein matrix comprises the polyelectrolyte, it is still possible that the protein matrix comprises one or several polyvalent ions, but such polyvalent ions should not be able to form a heat-resistant gel with the polyelectrolyte. Their nature and/or their concentration should not allow the formation of a heat-resistant gel with the polyelectrolyte.

Preferably, the step of preparing the protein matrix 130 further comprises an addition of the polyelectrolyte and the step of preparing the fatty matrix 140 further comprises an addition of the polyvalent ion, said polyelectrolyte and polyvalent ion forming a heat-resistant gel when combined.

In an embodiment, the protein matrix comprises said polyvalent ion and the fatty matrix comprises said polyelectrolyte. In a preferred embodiment, the protein matrix comprises said polyelectrolyte and the fatty matrix comprises said polyvalent ion.

In a preferred embodiment, the texturizing molecule comprises a polyelectrolyte and a polyvalent ion forming a heat-resistant gel when combined and also a cross-linking molecule. The cross-linking molecule can be in the fatty matrix, the protein matrix or both. Preferably the cross-linking molecule is selected among the ones described here before.

### Polyelectrolyte

As mentioned, the polyelectrolyte adapted to form the heat-resistant gel can be added to the protein matrix or the fatty matrix. A minimal quantity of polyelectrolyte can be of interest to improve the properties of the thread.

Hence, the protein matrix or the fatty matrix can comprise at least 0.01% in weight of polyelectrolyte compared to the total wet weight of the related matrix. Preferably, the protein matrix or the fatty matrix can comprise at least 0.02% in weight of polyelectrolyte compared to the total wet weight of the related matrix; more preferably at least 0.05% in weight, even more preferably at least 0.1% in weight, compared to the total wet weight of the related matrix.

In particular, when the protein matrix comprises the polyelectrolyte, said protein matrix can comprise at least 0.1% in weight of polyelectrolyte compared to the total wet weight of the protein matrix. Preferably, the protein matrix can comprise at least 0.2% in weight of polyelectrolyte compared to the total wet weight of the protein matrix; more preferably at least 0.3% in weight, even more preferably at least 0.4% in weight, compared to the total wet weight of the protein matrix. When the concentration of a polyelectrolyte is mentioned herein, it preferably refers to the concentration of the polyelectrolyte salt.

The protein matrix or the fatty matrix can comprise at most 15% in weight of polyelectrolyte compared to the total wet weight of the related matrix. Preferably, the protein matrix or the fatty matrix can comprise at most 10% in weight of polyelectrolyte compared to the total wet weight of the related matrix; more preferably at most 5% in weight, even more preferably at most 3% in weight, compared to the total wet weight of the related matrix. In particular as illustrated in examples, the protein matrix can comprise less than 2% in weight of polyelectrolyte compared to the total wet weight of the protein matrix. More particularly, the protein matrix comprises less than 2% in weight of alginate compared to the total wet weight of the protein matrix.

The protein matrix or the fatty matrix can comprise from 0.01% to 15% in weight of polyelectrolyte compared to the total wet weight of the related matrix. Preferably, the protein matrix or the fatty matrix can comprise from 0.02% to 10% in weight of polyelectrolyte compared to the total wet weight of the related matrix; more preferably from 0.05% to 5% in weight, even more preferably from 0.1% to 3% in weight, compared to the total wet weight of the related matrix.

As mentioned, the polyelectrolyte is a heat-resistant gel-forming polyelectrolyte. Preferably, also, the polyelectrolyte is not a heat-activable gel-forming polyelectrolyte. For example, the polyelectrolyte does not need a heat treatment at a temperature above 80° C., preferably above 60° C., to be activated.

Also, since it is intended for human consumption, the polyelectrolyte is digestible by the human digestive system.

For example, the polyelectrolyte is either cationic (e.g. chitosan) or anionic (e.g. alginate). Preferably, when the polyelectrolyte is an anionic polyelectrolyte then the polyvalent ion is a polyvalent cation. Also, when the polyelectrolyte is a cationic polyelectrolyte then the polyvalent ion is a polyvalent anion. More preferably, the polyelectrolyte is an anionic polysaccharide.

It is not necessary that all the monomers of the polyelectrolyte carry an ionisable group. For example, the polyelectrolyte is a polymer having at least 10% of its monomers bearing an ionisable group, preferably at least 20%, more preferably at least 30% and even more preferably at least 40%. Preferably, the ionisable group is selected from amine, carboxylic, sulphate, or phosphate groups.

For example, the polyelectrolyte is a polymer having at least 10% of its monomers that can be negatively charged, preferably at least 10%, more preferably at least 20%, even more preferably at least 30%, for example all the monomers can be negatively charged. Preferably, a monomer that can be negatively charged comprises at least one anionic group. Preferably, the anionic group is selected from carboxylic, sulphate, or phosphate groups, more preferably the anionic group is a carboxylic group.

For example, the polyelectrolyte is a polymer having at least 10% of its monomers that can be positively charged, preferably at least 20%, more preferably at least 30%, even more preferably at least 40%, for example all the monomers can be positively charged. Preferably, a monomer that can be positively charged comprises at least one cationic group. Preferably, the cationic group is an amine.

Preferably, the polyelectrolyte is a high molecular weight polyelectrolyte. For example, the polyelectrolyte has an average molar mass of at least 2 kg·mol⁻¹. Preferably, the polyelectrolyte has an average molar mass of at least 10 kg·mol⁻¹; more preferably, the polyelectrolyte has an average molar mass of at least 20 kg·mol⁻¹; even more preferably the polyelectrolyte has an average molar mass of at least 30 kg·mol⁻¹.

For optimized texture, the polyelectrolyte molecular weight should not be too high. For example, the polyelectrolyte has an average molar mass of at most 3000 kg·mol⁻¹. Preferably, the polyelectrolyte has an average molar mass of at most 2500 kg·mol⁻¹; more preferably, the polyelectrolyte has an average molar mass of at most 2000 kg·mol⁻¹; even more preferably the polyelectrolyte has an average molar mass of at most 1500 kg·mol⁻¹.

Hence, for example, the polyelectrolyte has an average molecular mass from 2 kg·mol⁻¹ to 3000 kg·mol⁻¹. Preferably, the polyelectrolyte has an average molar mass from 10 kg·mol⁻¹ to 2500 kg·mol⁻¹; more preferably, the polyelectrolyte has an average molar mass from 20 kg·mol⁻¹ to 2000 kg·mol⁻¹; even more preferably the polyelectrolyte has an average molar mass from 30 kg·mol⁻¹ to 1500 kg·mol⁻¹.

The average molecular weight of the polyelectrolyte can be measured by size exclusion chromatography coupled with light scattering. Preferably, it can be measured by following the instructions of the international norm ISO 16014-5:2019.

The present invention can use several polyelectrolytes. However, some polyelectrolytes are preferred. Preferably, the polyelectrolytes are selected from: low methoxyl pectin, low methoxyl pectin derivatives, high methoxyl pectin, high methoxyl pectin derivatives, alginate, alginate derivatives, xanthan, xanthan derivatives, chitosan, chitosan derivatives, ionic carboxymethyl-cellulose derivatives, ionic pullulan derivatives, ionic dextran derivatives, ionic starch derivatives, or a combination thereof or a combination thereof.

More preferably the polyelectrolyte is selected from alginate, alginate derivatives, low methoxyl pectin, low methoxyl pectin derivatives, chitosan, chitosan derivatives or combination thereof.

Even more preferably the polyelectrolyte is selected from alginate, low methoxyl pectin, chitosan, or combination thereof.

### Polyvalent Ion

As mentioned, in a method according to the invention, the polyvalent ion combines with the polyelectrolyte to form a heat-resistant gel.

The polyvalent ion can be either polyvalent anion or a polyvalent cation. For example, the polyvalent ion is selected among: calcium, magnesium, iron, manganese, copper, zinc, carbonate, oxalate, sulfate, sulfite, phosphate or combination thereof. Preferably, the polyvalent ion is selected among: calcium, magnesium, iron, manganese, copper, zinc, carbonate, oxalate, sulfate, phosphate or combination thereof.

The polyvalent ion can be obtained by dissolving the form of a salt. For example, the counter ions can be selected among: calcium, magnesium, sodium, ammonium, chloride, gluconate, lactate, sulfate, carbonate, phosphate, or combination thereof. When the concentration of a polyvalent ion is mentioned herein, it preferably refers to the concentration of the polyvalent ion as such.

The polyvalent ion is present in either the protein matrix or the fatty matrix. It can be present at a concentration of at least 0.001% in weight compared to the wet weight of the matrix, preferably at least 0.002%, more preferably at least 0.005% and even more preferably at least 0.01%, compared to the wet weight of the matrix.

The maximum concentration of the polyvalent ion is usually directed by the solubility of the polyvalent ion. For example, the polyvalent ion can be present in either the protein matrix or the fatty matrix at a concentration of at most 3% in weight compared to the wet weight of the matrix, preferably at most 2%, more preferably at most 1% and even more preferably at most 0.5%, compared to the wet weight of the matrix.

Hence, the polyvalent ion can be present in either the protein matrix or the fatty matrix at a concentration from 0.001 to 3% in weight compared to the wet weight of the matrix. Preferably, the polyvalent ion is present in either the protein matrix or the fatty matrix at a concentration from 0.002 to 2% in weight compared to the wet weight of the matrix; more preferably from 0.005 to 1% in weight, even more preferably from 0.01 to 0.5% in weight, compared to the wet weight of the matrix.

### Supplements

As mentioned in the example, the presence of supplemented fat, such as non-animal fat, in the protein matrix can improve the properties of the produced protein threads. Hence, advantageously, the step of preparation of the protein matrix further comprises an addition of fat, such as non-animal fat. Preferably, the supplemented fat is plant fat, or fermentation-obtained fat.

For example, the protein matrix can comprise at least 1% in weight of supplemented fat, preferably plant fat or fermentation-obtained fat, compared to the total wet weight of the protein matrix. Preferably, at least 2% in weight of supplemented fat compared to the total wet weight of the protein matrix, more preferably at least 5% in weight, even more preferably at least 10% in weight, compared to the total wet weight of the protein matrix. However, the quantity of supplemented fat should not be too high. For example, the protein matrix can comprise at most 50% in weight of supplemented fat compared to the total wet weight of the protein matrix. Preferably, at most 40% in weight of supplemented fat compared to the total wet weight of the protein matrix, more preferably at most 30% in weight, even more preferably at most 25% in weight, compared to the total wet weight of the protein matrix.

Hence, the protein matrix can comprise from 1% to 50% in weight of supplemented fat compared to the total wet weight of the protein matrix. Preferably, from 2% to 40% in weight of supplemented fat compared to the total wet weight of the protein matrix, more preferably from 5% to 30% in weight, even more preferably from 10% to 25% in weight, compared to the total wet weight of the protein matrix.

For example, the protein matrix can further comprise plant fat, preferably the plant fat comprise fat or oil extracted from edible plant matter, including the flowers, fruits, stems, leaves, roots, germs and seeds, more preferably, the plant fat comprises fat or oil extracted from oilseeds or fruits; in particular, the plant fat comprises fat extracted from canola seed (rapeseed), castor, coconut, flaxseed, allanblackia, olive, sunflower, soybean, peanut, illipe, cottonseed, shea, palm, avocado, safflower, sesame, lemon, grapeseed, macadamia, almond, sal, kokum, or mango or a combination thereof.

In particular, the plant fat used according to the invention can be selected from olive oil, palm oil, sunflower oil, avocado oil, almond oil or combination thereof.

The fermented fat can comprise fat or oil extracted from cells cultivated in anaerobic or aerobic fermentation process, in particular process involving cultivation of oleaginous microorganisms or animal cells excluding human cells. For example, the fermented fat can include fat from cyanobacteria, microalgae, yeast, fungi such as filamentous fungi, bacteria or cultivated animal cells excluding human cells, such as adipocytes. Preferably, the fermented fat comprises fat or oil extracted from oleaginous yeast, such as Rhodosporidium toruloides, Lipomyces starkeyi and Yarrowia lipolytica.

The protein matrix and the fatty matrix can undergo various preparations to improve the efficiency of protein threads formation. The drawings and the present description should not be understood as limiting the invention to an embodiment wherein the protein matrix is prepared before the fatty matrix. The person skilled in the art knows that, within the scope of the invention, the fatty matrix can also be prepared before the protein matrix or concomitantly.

The steps of preparation of the protein matrix or the fatty matrix can comprise a step of modification of the pH of any of said matrices. For example, the protein matrix has a pH value between 2 and 12, preferably 3 and 11, more preferably between 4 and 10, even more preferably between 5 and 9. For example, the fatty matrix has a pH value between 2 and 12, preferably 3 and 11, more preferably between 4 and 10, even more preferably between 5 and 9.

As it has already been mentioned, the temperatures at which the matrices are prepared and used in the context of the invention can have a marked effect on the quality of the protein threads produced.

Preferably, the protein matrix is not heated at a temperature of 65° C. or above for a duration of more than 10 minutes, preferably the protein matrix is not heated at a temperature of 60° C. or above, more preferably of 55° C. or above, even more preferably of 50° C. or above; for a duration of more than 10 minutes.

For example, the temperature of the protein matrix during its preparation step is lower than 65° C., preferably lower than 60° C., more preferably lower than 55° C., even more preferably lower than 50° C.

Preferably, the fatty matrix is not heated at a temperature above 85° C. for a duration of more than 10 minutes, preferably above 75° C., more preferably above 65° C., even more preferably above 55° C.; for a duration of more than 10 minutes.

For example, the temperature of the fatty matrix during its preparation step is at most 85° C., preferably at most 75° C. more preferably at most 65° C., even more preferably at most 55° C.

The protein matrix and the fatty matrix can also undergo physical treatment such as blending, mixing, homogenizing, sieving, or sonication. Preferably, the steps of preparation of the protein matrix or the fatty matrix can comprise a blending or homogenizing or emulsifying or stirring steps. These steps are in particular designed to homogenize or to mix all the components together. Also, these steps can result in a, at least partial, disruption of the cultivated non-human animal cells, when present. The steps of preparation of the protein matrix can comprise a disruption of the membrane of the non-human animal cells. Hence, the homogenizing step can be a blending step. It can for example be used when the protein matrix or the fatty matrix has been supplemented with other ingredients such as plant material or food additives. Preferably, the homogenizing or blending or emulsifying, or stirring step is performed with a high-speed mixer, or with a homogenizer, as a rotor-stator homogenizer, a cutter or a colloid mill.

The homogenizing may be conducted during at least 30 seconds, preferably at least 1 min, more preferably at least 2 min; for example, at least 10 min. The homogenizing step may be conducted during at most one hour, preferably at most 45 min, more preferably at most 30 min; even more preferably at most 10 min; for example, at most 2 min. Hence, the homogenizing may be conducted during 30 sec to 60 min, preferably during 1 min to 45 min, more preferably during 2 min to 30 min; for example during about 10 minutes.

The homogenizing may be conducted at least at 100 rpm, preferably at least 1000 rpm, more preferably at least 2000 rpm; for example, at least 5000 rpm. The homogenizing may be conducted during at most at 30 000 rpm, preferably at most 25 000 rpm, more preferably at most 20 000 rpm; for example, at most at 15 000 rpm. Hence, the homogenizing may be conducted at 100 rpm to 30 000 rpm, preferably at 1000 rpm to 25 000 rpm, more preferably at 2 000 rpm to 20 000 rpm; even more preferably at 5 000 rpm to 15 000 rpm. The homogenization preferably induces an emulsion, more preferably a microemulsion in the protein matrix. Also, the method of the invention comprises an homogenization of the fatty matrix to create an oil in water emulsion in the fatty matrix.

The preparation of the protein matrix can preferably comprise a homogenization, preferably the homogenization is conducted at least at 100 rpm for a period of at least 30 seconds. The homogenization improves the properties of the edible protein thread embedded in the fatty matrix. Moreover, when combined with the fat addition it can be used to create an emulsion which can improve the properties of the edible protein threads.

As shown in FIG. 1, a method according to the invention can comprise a step of adding a food additive 150. The food additive can be added to the protein matrix, the fatty matrix or both. This step is in particular designed to improve the flavor, the texture, the appearance or the shelf-life of the protein threads. The protein matrix or fatty matrix may comprise between 0.01 and 25% in weight of food additive, preferably between 0.01 and 10% in weight of food additive compared to the total wet weight of the according matrix. In turn, the protein threads may comprise between 0.01 and 10% in weight of food additive compared to the total weight of the protein threads, preferably between 0.01 and 4% in weight of food additive compared to the total weight of the protein threads.

The food additive can be selected from seasoning, flavoring additive, texturizing additive, food colorant, preservative additive or a combination thereof.

A food additive can be for example seasoning, mineral salt, flavoring additive, humectant additive, texturizing additive, anti-foaming agent, emulsifying additive, firming agent, gelling agent, stabilizing additive, thickening agent, food colorant, preservative additive or a combination thereof.

A seasoning can for example be selected from: salt; pepper; aromatic herbs and/or spices, including rosemary, sage, mint, oregano, parsley, thyme, bay leaf, cloves, basil, chives, marjoram, nutmeg, cardamom, chiles, cinnamon, fennel, fenugreek, ginger, saffron, vanilla and coriander; alcohol, including wine such as jurangon, sauterne or pacherenc, spirituous such as cognac or armagnac; or any combination thereof.

A flavoring additive can for example be selected from: flavor enhancer, sweetener, or any combination thereof.

A texturizing additive can for example be selected from: bulking agent or thickener, desiccant, curing agent or any combination thereof. For example, the protein matrix can further comprise a texturizing additive such as carrageenan, pectin, pullulan, dextran, starch.

Preferably, and as illustrated in examples, the fatty matrix can further comprise a hydrocolloid such as methylcellulose or gums.

A preservative additive can for example be selected from: antimicrobial agent, pH modulator, or any combination thereof.

A food colorant can for example be selected from colorants extracted from natural sources such as carotenes, anthocyanins, tomato (lycopene), beetroot (betacyanins and betaxanthins), or a mixture of thereof. The edible food product may comprise between 0.01% and 4% of food colorants compared to the total weight of the edible food product.

In particular, in a first embodiment, as it is illustrated in examples, the protein matrix can comprise:
at least 5% in weight of total proteins, preferably at least 10% in weight of total proteins, with respect to the total wet weight of the protein matrix, said total proteins including at least 5% of non-human animal proteins with respect to the total proteins weight, and
at least one texturizing molecule.

Preferably, as it is illustrated in examples, the protein matrix can comprise:
at least 5% in weight of total proteins, preferably at least 10% in weight of total proteins, with respect to the total wet weight of the protein matrix, said total proteins including at least 5% of non-human animal proteins with respect to the total proteins weight, and
at least 0.1% in weight of the polyelectrolyte with respect to the total wet weight of the protein matrix.

Preferably, as it is illustrated in examples, the protein matrix can comprise:
at least 5% in weight of total proteins, preferably at least 10% in weight of total proteins with respect to the total wet weight of the protein matrix, said total proteins including at least 5% of non-human animal proteins with respect to the total proteins weight, and
at least 0.1% in weight of the polyelectrolyte with respect to the total wet weight of the protein matrix, and
at least 1% of plant fat or fermentation-obtained fat, with respect to the total wet weight of the protein matrix

Preferably, as it is illustrated in examples, the protein matrix can comprise:
at least 5% in weight of total proteins, preferably at least 10% in weight of total proteins with respect to the total wet weight of the protein matrix, said total proteins including at least 5% of non-human animal proteins with respect to the total proteins weight, and
at least 0.1% in weight of the polyelectrolyte with respect to the total wet weight of the protein matrix, and
at least 1% of plant fat or fermentation-obtained fat, with respect to the total wet weight of the protein matrix, and
at least one cross-linking molecule.

Even more preferably, the protein matrix can comprise:
at least 5% in weight of total proteins, preferably at least 10% in weight of total proteins with respect to the total wet weight of the protein matrix, said total proteins including at least 5% of non-human animal proteins with respect to the total proteins weight, and
at least 0.1% in weight of the polyelectrolyte with respect to the total wet weight of the protein matrix;
at least one cross-linking molecule.

In particular, in a second embodiment, the protein matrix can comprise:
at least 5% in weight of total proteins, preferably at least 10% in weight of total proteins, with respect to the total wet weight of the protein matrix, said total proteins including at least 5% of non-human animal proteins with respect to the total proteins weight, and
at least 0.001% in weight of the polyvalent ion with respect to the total wet weight of the protein matrix.

Preferably, the protein matrix can comprise:
at least 5% in weight of total proteins, preferably at least 10% in weight of total proteins with respect to the total wet weight of the protein matrix, said total proteins including at least 5% of non-human animal proteins with respect to the total proteins weight, and
at least 0.001% in weight of the polyvalent ion with respect to the total wet weight of the protein matrix, and
at least 1% of plant fat or fermentation-obtained fat, with respect to the total wet weight of the protein matrix.

More preferably, the protein matrix can comprise:
at least 5% in weight of total proteins, preferably at least 10% in weight of total proteins with respect to the total wet weight of the protein matrix, said total proteins including at least 5% of non-human animal proteins with respect to the total proteins weight and at least 0.25% in weight of myofibrillar proteins compared to the total proteins weight, and
at least 0.001% in weight of the polyvalent ion with respect to the total wet weight of the protein matrix.

Even more preferably, the protein matrix can comprise:
at least 5% in weight of total proteins, preferably at least 10% in weight of total proteins with respect to the total wet weight of the protein matrix, said total proteins including at least 5% of non-human animal proteins with respect to the total proteins weight,
at least 0.001% in weight of the polyvalent ion with respect to the total wet weight of the protein matrix, and
at least one cross-linking molecule.

In particular, in a first embodiment, as it is illustrated in examples, the fatty matrix can comprise:
at least 20% in weight of fat, preferably at least 40% in weight in fat, with respect to the total wet weight of the fatty matrix, and
at least one texturizing molecule.

Preferably, the fatty matrix can comprise:
at least 20% in weight of fat, preferably at least 40% in weight in fat, with respect to the total wet weight of the fatty matrix, and
at least 0.001% in weight of the polyvalent ion with respect to the total wet weight of the fatty matrix, and
at least one cross-linking molecule.

More preferably, as it is illustrated in examples, the fatty matrix can comprise:
at least 20% in weight of fat, preferably at least 40% in weight in fat, with respect to the total wet weight of the fatty matrix,
at least 0.25% of proteins in weigh, preferably at 0.50% of proteins in weight, with respect to the total weight of fatty matrix, and
at least 0.001% in weight of the polyvalent ion with respect to the total wet weight of the fatty matrix.

Even more preferably, as it is illustrated in examples, the fatty matrix can comprise:
at least 20% in weight of fat, preferably at least 40% in weight in fat, with respect to the total wet weight of the fatty matrix,
at least 0.25% of proteins in weight, preferably at 0.50% of proteins in weight, with respect to the total wet weight of the fatty matrix,
at least 0.001% in weight of the polyvalent ion with respect to the total wet weight of the fatty matrix, and
at least one cross-linking molecule.

Even more preferably, as it is illustrated in examples, the fatty matrix can comprise:
at least 20% in weight of fat, preferably at least 40% in weight in fat, with respect to the total wet weight of the fatty matrix, said fat comprising at least 60% in weight of triglycerides compared to the total weight of the fat of the fatty matrix, and
at least 0.25% of proteins in weight, preferably at 0.50% of proteins in weight, with respect to the total wet weight of the fatty matrix, and
at least one texturizing molecule.

In particular, in a second embodiment, the fatty matrix can comprise:
at least 20% in weight of fat, preferably at least 40% in weight in fat, with respect to the total wet weight of the fatty matrix, and
at least 0.01% in weight of the polyelectrolyte with respect to the total wet weight of the fatty matrix.

Preferably, the fatty matrix can comprise:
at least 20% in weight of fat, preferably at least 40% in weight in fat, with respect to the total wet weight of the fatty matrix,
at least 0.01% in weight of the polyelectrolyte with respect to the total wet weight of the fatty matrix, and
at least one cross-linking molecule.

Preferably, the fatty matrix can comprise:
at least 20% in weight of fat, preferably at least 40% in weight in fat, with respect to the total wet weight of the fatty matrix,
at least 0.25% of proteins in weight, preferably at 0.50% of proteins in weight, with respect to the total wet weight of the fatty matrix,
at least 0.01% in weight of the polyelectrolyte with respect to the total wet weight of the fatty matrix, and
at least one cross-linking molecule.

As shown in FIG. 1, a method 100 of manufacturing an edible food product according to the invention comprises a step of contacting 160 the protein matrix with the fatty matrix. This step is in particular designed to form an edible protein thread embedded in the fatty matrix. More particularly, several edible protein threads that can be prepared concomitantly.

Advantageously, as illustrated in the examples, the temperature of the protein matrix when contacting the fatty matrix is lower than 65° C. Preferably, the temperature of the protein matrix when contacting the fatty matrix is lower than 60° C., more preferably lower than 55° C., even more preferably lower than 50° C. This can improve the texture, the operability, and/or the appearance of the edible protein threads.

Moreover, once prepared, the protein matrix should not undergo a heat treatment at a temperature of 65° C. or higher, preferably of 60° C. or higher, more preferably of 55° C. or higher, even more preferably of 50° C. or higher.

For example, the temperature of the protein matrix used during the contacting 160 step, is between 1° C. and 65° C., preferably between 3° C. and 60° C., more preferably between 3° C. to 55° C., even more preferably between 3° C. to 50° C.

It has been shown that the fatty matrix can be used at a higher temperature with no effect on the qualities of the produced edible food product. Hence, the temperature of the fatty matrix when contacting the protein matrix can be higher than 35° C. However, the temperature of the fatty matrix when contacting with the protein matrix should preferably be of at most 85° C., more preferably at most 75° C., even more preferably at most 65° C., for example at a temperature below 60° C.

Indeed, when the fat is heated prior to the preparation of the fatty matrix, the method according to the invention can comprise a cooling step of the fatty matrix before the step of contacting the protein matrix and the fatty matrix.

For example, the fat can be heated at a temperature of at least 40° C. to prepare the fatty matrix, at least 50° C., at least 60° C., or even 85° C. or higher. Before contacting the protein matrix with the fatty matrix, the method according to the invention preferably comprises a step of cooling the fatty matrix.

The fatty matrix can have, during the contacting step, a viscosity from 100 to 50000 Pa·s, when measured at 25° C. at a shear rate of 0.1 s⁻¹

The protein matrix can have, during the contacting step, a viscosity from 50 to 50000 Pa·s, when measured at 25° C. at a shear rate of 0.1 s⁻¹.

The fatty matrix and the protein matrix can have, during the contacting step, viscosities such that the ratio of the viscosity of the protein matrix to the viscosity of the fatty matrix is below 100, preferably below 50, more preferably from below 20, even more preferably below 10, for example below 1. In particular, the fatty matrix and the protein matrix can have, during the contacting step, viscosities such that the ratio of the viscosity of the protein matrix to the viscosity of the fatty matrix is from 0.005 to 100, preferably from 0.007 to 50, more preferably from 0.01 to 20.

The step of contacting 160 the protein matrix with the fatty matrix can be performed by known methods such as extrusion spinning, gel spinning, melt spinning, centrifugal spinning, bath-assisted 3D printing, microextrusion, dry spinning, wet spinning or a dry jet wet spinning. Preferably, extrusion spinning, gel spinning, melt spinning, centrifugal spinning, bath-assisted 3D printing, wet spinning or a dry jet wet spinning. More preferably, the step of contacting 160 the protein matrix with the fatty matrix is performed by wet spinning or a dry jet wet spinning, bath-assisted 3D printing. Even more preferably, the step of contacting 160 the protein matrix with the fatty matrix is performed by wet spinning or a dry jet wet spinning.

In particular, the step of contacting 160 the protein matrix with the fatty matrix can comprise impregnating the protein matrix with the fatty matrix and preferably immersing the protein matrix, preferably in the form of a thread, in a bath made of the fatty matrix.

Preferably, the step of contacting 160 the protein matrix with the fatty matrix comprises an implementation of a movement of the protein matrix or the fatty matrix with respect to the other one. In particular, the movement is implemented at a contacting point between the protein matrix and the fatty matrix. The contacting point can correspond to an injection site as it will be described hereafter.

For example, a movement could be created in the fatty matrix (for example through a vortex collector). Such a movement can help elongate the edible protein threads in formation.

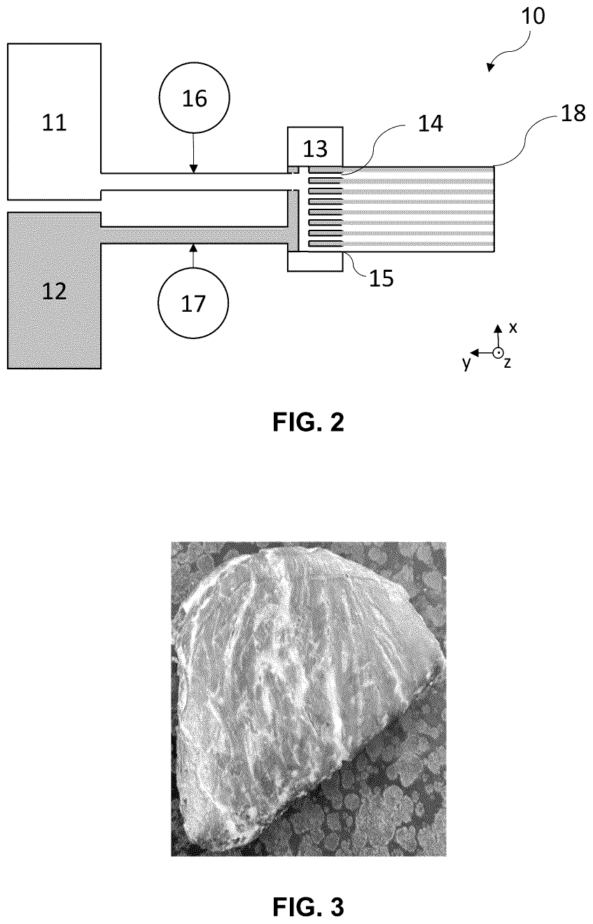

A movement can also be implemented on the means used to introduce the protein matrix in the fatty matrix. For instance, as it will be described hereafter and in connection with FIG. 2, the method can comprise a movement of the needles used to spin the protein matrix along the three axes (x,y,z).

Alternatively, a movement can also be implemented on the means comprising the fatty matrix. For instance, as it will be described hereafter, the method can comprise a movement of a container comprising the fatty matrix or stirring means inside the container comprising the fatty matrix. Moreover, the method can comprise a movement of a production container comprising the fatty matrix or stirring means inside the production container comprising the fatty matrix.

In particular, the step of contacting 160 comprises an immersion of the protein matrix in the fatty matrix and the formation of the edible protein threads in a bath made of the fatty matrix.

The step of contacting 160 the protein matrix with the fatty matrix can be performed during at least 10 ms, preferably during at least 100 ms, more preferably during at least 500 ms, even more preferably during at least 1 second.

The step of contacting 160 the protein matrix with the fatty matrix can be performed during at most 20 minutes, preferably during at most 10 minutes, more preferably during at most 5 minutes, even more preferably during at most 4 minutes.

Hence, the step of contacting 160 the protein matrix with the fatty matrix can be performed during from 10 ms to 20 min preferably during from 100 ms to 10 minutes more preferably during from 500 ms to 5 minutes, even more preferably during from 1 second to 4 minutes.

The contacting 160 step can be followed by a cooling step. Indeed, in some embodiments the fatty matrix can be heated prior and/or during the contacting step. Hence, the method can comprise a step of cooling the protein threads embedded in the fatty matrix, thus cooling the edible food product produced by the method according to the invention.

Preferably, the cooling step is conducted at a temperature of at most 10° C. More preferably, the cooling step is conducted at a temperature of at most 5° C.

Preferably the cooling step is initiated at most 10 minutes after the contacting step, preferably at most 5 min after the contacting step.

As shown in FIG. 1, a method according to the invention can comprise a step of transforming 170 the protein threads embedded into the fatty matrix.

This step is in particular designed to prepare the edible protein threads embedded into the fatty matrix so that they are suitable for the subsequent steps, in particular for the conditioning of an edible food product which can be an edible meat substitute. Also, the step of transforming the edible protein threads embedded into the fatty matrix can also comprise a combination of the edible protein threads embedded into the fatty matrix with another food matrix, such as a carbohydrate matrix.

The assembling to the protein threads can comprise assembling protein threads into a structure that mimics the texture of meat. The process includes collecting and aligning the protein threads, and further processing them through various treatments to produce a meat-like texture and consistency.

In one embodiment of the present invention, the assembly of the protein threads into a meat-like texture comprises one or several of the following steps which can be carried out in any different order:
Collecting and stretching the protein threads to orient the proteins longitudinally, similarly to the muscle fiber orientation in meat;
Organizing the protein threads in parallel or entangled configurations, followed by a lamination process where the threads are compressed;
Layering the protein threads upon one another, each layer being positioned to contribute to the overall meat-like structure;
Cross-linking the protein threads either chemically and/or enzymatically and/or physically to enhance the textural properties and integrity of the assembled protein structure;
Applying mild heat and pressure to the protein threads to improve protein-protein interactions; and/or
Weaving the protein threads into a fabric-like structure using a loom, designed to replicate the interwoven nature of muscle fibers in meat;
Also, when assembling the edible protein threads, it will be relevant to use a binder-like composition such as a fatty matrix and/or connective tissue analogues to improve the mechanical and organoleptic properties of the edible product.

For example, the embedded protein threads can undergo further processing such as marination, crushing, squishing, braiding, cutting, mincing, grinding, mixing, shredding, squeezing, dosing, molding, pressing, 3D printing, extruding baking, cooling, freezing or cooking steps such as smoking, roasting, frying, surface treatment, coating or also combination with other ingredients, in order to produce an edible food product which mimics known conventional meat products. In particular, the edible product can be an alternative product to the meat which aims to imitate a known meat product, transformed or not.

As shown in FIG. 1, a method according to the invention can comprise a step of conditioning 180 an edible food product comprising the edible protein threads embedded into the fatty matrix.

This step is in particular designed to obtain an edible food product having meat-like properties, such as meat-like organoleptic properties in particular meat-like texture or flavor.

According to the invention, the step of conditioning 180 the edible food product can comprise steps such as steps that affect water content, shape, texture, flavor or even shelf life of the edible food.

For example, according to the invention, the step of conditioning 180 the edible food product can comprise steps such as drying, desiccation, dehydration, lyophilization, filtering or a combination thereof.

For example, according to the invention, the step of conditioning 180 the edible food product can comprise steps such as sterilization or pasteurization.

Finally, according to the invention, the step of conditioning 180 the edible food product can comprise steps such as cooling, refrigeration, deep-freezing, packaging or a combination thereof.

In another aspect, the invention relates to an edible food product obtainable from a method according to the invention. Preferably, the edible food product comprising edible protein threads embedded into the fatty matrix. More preferably, the edible food product has been obtained by a method according to the invention.

In particular, the proteins of the edible protein threads are selected among plant proteins, microbial proteins, algae proteins, animal proteins, fungal proteins, and a combination thereof. Preferably, the proteins of the edible protein threads comprise at least non-human animal proteins, said non-human animal proteins being cultivated non-human animal cell proteins. Advantageously, this edible protein thread has an improved meat-like flavor and/or meat-like texture compared to edible protein thread made from only plant proteins.

The invention preferably relates to an edible food product obtainable from a method according to the invention, said edible food product comprising edible protein threads being embedded into the fatty matrix.

Furthermore, this edible protein thread is preferably a heat-resistant protein thread, said heat-resistant protein thread being formed from one or more texturizing molecule(s) capable of creating a heat-resistant thread. More preferably, the edible protein thread comprises a polyelectrolyte combined with a suitable polyvalent ion to form the heat-resistant protein thread.

Several embodiments, whether preferred or not preferred, have been described here before in connection to the method according to the invention to produce the edible food product of the invention. Hence, the edible food product according to the invention (e.g. protein threads and fatty matrix) may comprise, alone or in combination, each of the features described above in connection with a method according to the invention and any of its above-mentioned steps.

An edible protein thread embedded in the fatty matrix according to the invention can have been produced from a cross-linking molecule used either in the protein matrix and/or the fatty matrix. Preferably, an edible protein thread embedded in the fatty matrix according to the invention can have been produced from a protein matrix comprising one of either the polyvalent ion or the polyelectrolyte, and a fatty matrix which comprises the other. In particular, an edible protein thread embedded in the fatty matrix according to the invention can have been produced from a protein matrix comprising the polyelectrolyte or from a protein matrix comprising the polyvalent ion. In an embodiment, the fatty matrix and/or the protein matrix further comprise a cross-linking molecule, in addition to the polyelectrolyte or the polyvalent ion.

Advantageously, the edible protein thread embedded in the fatty matrix has been produced from non-human cultivated animal cells. Hence, as it is detailed hereafter, the edible protein thread embedded in the fatty matrix according to the invention can comprise intact cultivated cells, disrupted cultivated cells and/or extracts of cultivated cells (such as extracts of disrupted cultivated cells), said cells being cultivated cells from an organism of the Animalia kingdom excluding human. However, considering the preferred embodiments of the method according to the invention to produce the edible protein threads, the edible protein thread embedded in the fatty matrix may not contain intact cultivated cells.

An edible food product comprising edible protein threads embedded in the fatty matrix can comprise a ratio in weight of Fatty matrix on protein threads from 0.20 to 1.8, preferably from 0.25 to 1.6, more preferably from 0.3 to 1.4 and even more preferably from 0.4 to 1.2.

The edible protein thread embedded in the fatty matrix can comprise at least 0.25% in weight of myofibrillar proteins compared to the total weight of proteins, preferably at least 0.5% in weight of myofibrillar proteins, more preferably at least 1% in weight of myofibrillar proteins, even more preferably at least 2% wet weight of myofibrillar proteins compared to the total weight of proteins in the protein thread.

The edible protein thread embedded in the fatty matrix can comprise at most 70% in weight of myofibrillar proteins compared to the total weight of proteins, preferably at most 60% in weight of myofibrillar proteins, more preferably at most 50% in weight of myofibrillar proteins, even more preferably at most 40% in weight of myofibrillar proteins compared to the total weight of proteins in the protein thread.

Hence, the edible protein thread embedded in the fatty matrix can comprise between 0.25% and 70% in weight of myofibrillar proteins compared to the total weight of proteins, preferably between 0.5% and 60% in wet weight of myofibrillar proteins, more preferably between 1% and 50% in wet weight of myofibrillar proteins, even more preferably between 2% and 40% in wet weight of myofibrillar proteins compared to the total weight of proteins in the protein thread.

The concentration of myofibrillar proteins can also be considered regarding the concentration of polyelectrolyte. This ratio can be controlled to optimize the behavior of the protein threads. Hence, the edible protein thread embedded in the fatty matrix can comprise myofibrillar proteins (from cultivated cells) and polyelectrolyte at concentrations so that a ratio of myofibrillar proteins weight on polyelectrolyte is of at least 2.5, preferably 5, more preferably 7.5, even more preferably 10.

However, the edible protein thread embedded in the fatty matrix can comprise myofibrillar proteins and polyelectrolyte at concentrations so that a ratio of myofibrillar proteins weight on polyelectrolyte weight is of at most 100, preferably at most 50, more preferably at most 40, even more preferably at most 30.

Hence, the edible protein thread embedded in the fatty matrix can comprise myofibrillar proteins and polyelectrolyte at concentrations so that a ratio of myofibrillar proteins weight on polyelectrolyte weight is from 2.5 to 100, preferably from 5 to 50, more preferably from 7.5 to 40, even more preferably from 10 to 30. As mentioned, myofibrillar proteins can be quantified by mass spectrometry like the polyelectrolyte.

The edible protein thread embedded in the fatty matrix can comprise at least 0.1% in weight of polyelectrolyte compared to the total wet weight of the protein thread. Preferably, the edible protein thread embedded in the fatty matrix can comprise at least 0.2% in weight of polyelectrolyte compared to the total wet weight of the protein thread; more preferably at least 0.3% in weight, even more preferably at least 0.4% in weight, compared to the total wet weight of the protein thread.

The edible protein thread embedded in the fatty matrix can comprise at most 15% in weight of polyelectrolyte compared to the total wet weight of the protein thread. Preferably, the edible protein thread embedded in the fatty matrix can comprise at most 10% in weight of polyelectrolyte compared to the total wet weight of the protein thread; more preferably at most 5% in weight, even more preferably at most 3% in weight, compared to the total wet weight of the protein thread.

The edible protein thread embedded in the fatty matrix can comprise from 0.1% to 15% in weight of polyelectrolyte compared to the total wet weight of the protein thread. Preferably, the protein thread can comprise from 0.3% to 10% in weight of polyelectrolyte compared to the total wet weight of the protein thread; more preferably from 0.4% to 5% in weight, even more preferably from 0.5% to 3% in weight, compared to the total wet weight of the protein thread.

The edible protein thread embedded in the fatty matrix can comprise troponin protein preferably at least 0.0125% in weight of troponin protein compared to the total weight of proteins in the protein thread, preferably at least 0.025%, more preferably at least 0.05%, even more preferably at least 0.1%, compared to the total weight of proteins in the protein thread.

The edible protein thread embedded in the fatty matrix can comprise collagen, preferably at least 1% in weight of collagen compared to the total weight of proteins in the protein thread, preferably at least 2%, more preferably at least 3%, even more preferably at least 4%, compared to the total weight of proteins in the protein thread.

The edible protein thread embedded in the fatty matrix can comprise elastin and/or tropoelastin, preferably at least 0.065% in weight of elastin and/or tropoelastin compared to the total weight of proteins in the protein thread, preferably at least 0.13%, more preferably at least 0.2%, even more preferably at least 0.25%, compared to the total weight of proteins in the protein thread.

The edible protein thread embedded in the fatty matrix according to the invention has preferably a shape and/or mechanical properties that allow it to mimic the behavior of a conventional meat from a slaughtered animal when cooked and tasted.

For example, an edible protein thread embedded in the fatty matrix according to the invention has a length of at least 2 mm, preferably at least 10 mm, more preferably at least 50 mm, even more preferably at least 100 mm.

For example, an edible protein thread embedded in the fatty matrix according to the invention has a diameter of at most 4 mm, preferably at most 2 mm, more preferably at most 1 mm, even more preferably at most 0.5 mm.

Advantageously, the edible food product according to the invention can be considered as an ingredient for an alternative to conventional meat products from a slaughtered animal or an alternative to conventional meat products from a slaughtered animal as such.

An edible food product according to the invention can for example be a ready-to-eat food product that can be consumed directly, or eventually after a processing step (e.g. crushing, squishing, braiding, cutting, grinding, mixing, shredding, squeezing, dosing, molding, pressing, 3D printing, extruding baking or cooking steps such as smoking, roasting, frying, surface treatment, and/or coating) and/or a cooking step. An edible food product according to the invention can also be an intermediate product to be used in combination with other products to produce a ready-to-eat food product. In particular, the edible food product according to the invention can be an alternative product to the meat from a slaughtered animal which aims to imitate a conventional meat product (e.g. steak, sausage, pate . . . ). As it is illustrated in examples, an edible food product according to the invention can exhibit an improved meat-like texture and/or meat-like flavor compared to an edible meat alternative food product made from plant proteins.

As it has been mentioned, the edible food product according to the invention can be a finished product or an ingredient for food processing. Preferably, the edible food product according to the invention mimics an animal-derived edible food product. The edible food product according to the invention can be a cooked, a non-cooked or a precooked product.

For example, the edible food product according to the invention is a cooked edible food product or a pre-cooked edible food product. For example, the edible food product is precooked to be further pan-fried. Alternatively, the edible food product is a non-cooked product.

In another aspect, the invention relates to a system 10 for producing an edible food product comprising edible protein threads embedded in a fatty matrix.

As illustrated in FIG. 2, the system 10 according to the invention can comprise a protein matrix container 11 capable of containing a protein matrix, said protein matrix comprising proteins.

The protein matrix container 11 can be made of food grade plastics, ceramics, glasses, silicons, stainless steel, or combination thereof. The protein matrix container 11 can be associated with a temperature controller configured to measure and modify the temperature of the protein matrix. The protein matrix container 11 can be associated with a pressure controller configured to measure the pressure inside the protein matrix container.

As illustrated in FIG. 2, the system 10 according to the invention can comprise a fatty matrix container 12 capable of containing a fatty matrix.

The fatty matrix container 12 can be made of food grade plastics, ceramics, glasses, silicons, stainless steel, or combination thereof. The fatty matrix container 12 can be associated with a temperature controller configured to measure and modify the temperature of the fatty matrix.

As illustrated in FIG. 2, the system 10 according to the invention comprises a contacting device 13. Advantageously, the contacting device 13 is arranged to be able to bring in contact the protein matrix with the fatty matrix to form edible protein threads, from the protein matrix, embedded into the fatty matrix.

Advantageously, the contacting device 13 has one or several holes 14 connected to the protein matrix container 11, said one or several holes being arranged to allow the protein matrix to be brought into contact with the fatty matrix while being in the form of thread. Holes 14 can have an area lower than 4 mm², preferably lower than 3.5 mm², more preferably lower than 2 mm², more preferably lower than 1 mm², even more preferably lower than 0.5 mm². Holes can have an irregular or determined shape such as rectangular, round, oval . . . .

More preferably, the contacting device 13 is configured to be able to induce, during a contacting step, a displacement of the one or several holes 14. This can be used to improve the homogeneous aspect of the protein threads produced. In particular when the displacement of the one or several holes 14 is a displacement in relation to the fatty matrix.

For example, the contacting device 13 can comprise one or more needles 15 arranged to perform, preferably with a piston or a pumping device 16, an injection of the protein matrix into the fatty matrix. In particular, the one or more needles 15 are arranged to be mobile during the injection of the protein matrix into the fatty matrix. A system according to the invention can also comprise a piston or a pumping device 17 arranged to induce a movement of the fatty matrix.

In one embodiment, the contacting device 13 comprises multiple needles 15 connected to the pumping device 16 associated with the protein matrix container. Also, several needles 15 can be connected to a shared chamber or each needle 15 can be connected to an independent chamber. Said needles 15 could be arranged in parallel, one to each other, they can all display the same pattern (round, rectangular, oval, irregular) or present different patterns. Preferably, said needles 15 display different patterns in order to obtain an irregular section of the embedded protein threads into the fat, therefore providing a more natural aspect to the food product.

As illustrated in FIG. 2, the system 10 according to the invention can comprise a production container 18 capable of containing the protein threads embedded in the fatty matrix.

The production container 18 can be made of food grade plastics, ceramics, glasses, silicons, stainless steel, or combination thereof. The production container 18 can be associated with a temperature controller configured to measure and modify the temperature of the edible food product.

The production container 18 can take the shape of the final product, e.g. beef/pork striploan, beef/pork brisket, beef/pork tenderloin steak, salmon/tuna filet.

Advantageously, the production container 18 is arranged so as to allow the implementation of a movement of the fatty matrix, preferably a translational or rotational movement of the production container. This can be used to improve the homogeneous aspect of the protein threads produced.

Moreover, with such a system 10, the harvesting steps can be avoided and further processing steps can be reduced.

Preferably, the system 10 according to the invention can further comprise a cooling device. Preferably, the system 10 according to the invention can further comprise a cutting or a slicer device or a packaging device.

### EXAMPLES

The invention is further described in detail by reference to the following experimental examples. These examples are provided for purposes of illustration only and are not intended to be limiting unless otherwise specified. Thus, the invention should in no way be construed as being limited to the following illustrative examples, but rather, should be construed to encompass any and all variations which become evident as a result of the teaching provided herein.

Without further description, it is believed that one of ordinary skills in the art can, using the preceding description and the following illustrative examples, make and utilize the products of the present invention and practice the claimed methods. The following examples, therefore, specifically point out the preferred embodiments of the present invention, and are not to be construed as limiting in any way the remainder of the disclosure.

### Materials and Methods

### Chemicals

Sodium alginate food grade powder, E 401
Methylcellulose E461
Amylopectin n^{o} CAS 9037-22-3
Chitosan n^{o} CAS 9012-76-4
Calcium chloride n^{o} CAS 10043-52-4
NaCl
Transglutaminase (TGase) Powder—125 U/g
Elastin—Sigma
Recombinant collagen—Sigma

### Fat

Plant fat and/or fermented fat can be obtained through mechanical or chemical extraction from seeds or from other parts of fruit such as palm oil and avocado butter. It can then be purified and, if required, refined or chemically altered. Many commercial references can be used such as a mix of CremoFLEX® L and CremoFLEX® E. Alternatively, fermented fat produced by microalgae such as Bacillariophyceae, Chlorophyceae, Eustigmatophyceae, Rhodophyceae or Conjugatophyceae can be used. The chemical analysis, in particular triglycerides or fatty acid concentrations, of the fatty substances can be carried out using the analysis and quantification methods known to those skilled in the art (e.g. using ISO 12966).

### Cells Culture & Preparation

Bovine cells were bovine cells obtained from biopsies or cultivated cells. Cultivated cells can be bovine embryonic stem cells initially isolated from bovine embryos and adapted to grow in suspension in the serum free and growth factors free medium. These cells are characterized by their property to grow in suspension at a larger scale in bioreactors.

The bovine cells are cultivated in a 30 L stainless steel bioreactor at 37° C., pH 7.1 regulated with CO₂ injection under constant stirring at 50 rpm. Four days post seeding, cells are collected from the bioreactor, submitted to a two-step centrifugation and the dry pellets are weighed.

A protein dosage can be conducted using a Bradford method on a sample of the harvested cells.

The moisture content is measured and can be adjusted. Cells can be used as such. However, an additional step such as protein extraction can be performed on harvested cultivated cells.

### Protein Matrix Preparation

The protein matrix is prepared by mixing the ingredients following mix matrices.

The protein matrix is homogenized using a homogenizer (8000 rpm, 60 s).

### Fatty Matrix Composition & Preparation

The fatty matrix is prepared by mixing the ingredients following mix matrices hereunder described. Fats are added either liquid, solid or both.

The fatty matrix is homogenized using a homogenizer (8000 rpm, 60 s).

### Contacting the Protein Matrix and Fatty Matrix to Produce the Edible Food Product

Protein threads are spinned using a syringe pump (Chemyx), a 10 mL syringe equipped with 18 G (0.8 mm) or 14 G (1.6 mm) needle or a spinneret comprising several holes in the fatty matrix. The injection rate is fixed at 5 mL/min.

### Mechanical Test

Protein threads embedded in the fatty matrix are evaluated by tensile testing using a texturometer (Ametek LS1 equipped with a 10 kg load cell). Briefly, protein threads are subjected to an elongation until failure and the force is recorded as a function of the elongated distance. At failure, the measured force drops sharply.

The extension rate is 100 mm/min and measurements are performed on 6 samples for each diameter.

### Texture Profile Analysis

A texture profile analysis can be conducted to compare the texture of an edible meat substitute comprising edible protein threads and a fatty matrix having controlled fat marbling according to the invention to the texture of other food products.

Typically, each sample is cut into 2 cm square cubes, then pan-fried on a heating plate at 200° C. for 2 min per side until reaching a core temperature of 55±2° C. TPA measurements (6 repetitions per product) are performed using a texturometer equipped with a 500 N load cell and a compression plate probe of 75 mm of diameter.

Samples are subjected to two cycles of compression to 75% of their height with 10 s elapsed time. Measurements are performed at 20° C. and the compression direction is parallel to the fiber's orientation.

### Fat Release Properties

The fat release phenomenon during cooking and chewing is important when evaluating the textural properties of meat products, especially high fat content meat products such as marbled beef. Also, the fat release phenomenon can be correlated to the fat-explosion and mouthfeel that consumers experience while chewing.

As an edible food product according to the invention comprises edible protein threads and a fatty matrix having controlled fat marbling, a way of evaluating its ability to mimic the behavior of a conventional food product is to evaluate the quantity of leaked fat during a pan-fried style cooking.

Fat release measurements are conducted to compare the leaked fat. Each sample is cut into 2 cm square cubes, then pan-fried on a heating plate at 200° C. for 2 min per side until reaching a core temperature of 55±2° C.

The fat release value at cooking is preferably the mean value obtained from at least 5 independent samples. For example, fat release can be calculated by measuring the weight of the sample before and after cooking. The difference of weight of the samples before and after pan-frying equals the weight of fat released during cooking.

The capacity of the samples to release fat during cooking can be expressed in w/w % using the following equation:

$$( ( sample weight before cooking - sample weight after cooking ) / sample weight before cooking ) * 100$$

The fat release value after compression is preferably the mean value obtained from at least 5 independent samples. For example, fat release can be calculated by measuring the weight of the sample before and after the double compression test. Samples are subjected to two cycles of compression to 75% of their height with 10 s elapsed time. Measurements are performed at 20° C. and the compression direction is parallel to the fiber's orientation.

The difference of weight of the samples before and after pan-frying equals the weight of fat released during compression. This value correlates with sensory perception by the panelist to the fat release in mouth when consuming the edible food product.

The capacity of the samples to release fat during compression can be expressed in w/w % using the following equation:

((sample weight before compression−sample weight after compression)/sample weight before compression)*100

### Sensory Evaluations

Food samples are anonymized before tasting. Additionally, panelists are offered water for rinsing their mouth between samples and a cracker, to reset flavor receptors.

Panelists evaluate the overall liking and meat-like organoleptic properties of the edible food product samples by tasting. For example, to evaluate beef substitutes, panelists assess beef-meat appearance, flavor and texture (tenderness, cohesiveness, oiliness and juiciness).

### Influence of the Composition of the Fatty Matrix on the Edible Food Product

The fatty matrix allows the formation of the protein thread and also plays the role of a fatty phase in the final food product. Hence the fatty matrix needs to interact effectively with the protein matrix and should reproduce a meat-like texture, in particular the fatty part of meat, which is complex.

The protein matrix is prepared by mixing: 17.6% animal proteins from cells or cell extracts wt %; 10% liquid oil; 1% NaCl; 1% sodium alginate; and 70.4% water.

The fatty matrix is prepared by mixing the ingredients following table 1.

The protein matrix is used in a spinneret to induce a flow of the protein matrix coming into contact with the fatty matrix used as a coagulation bath.

Table 1 below shows compositions of the fatty matrix which either may (invention) or may not (comparative) solve the technical problem addressed by the present invention. In particular, different concentrations of proteins, fats and triglycerides are tested and the effect of these concentrations on the protein thread formation and texture of the food product is evaluated in order to find the ones having the consumer's approval.

TABLE 1
Quantity used for each of the preparations of the fatty matrix.

| Ingredients | | | | | | |
|---|---|---|---|---|---|---|
| (% w wet fatty | A1 | A2 | A3 | A4 | A5 | A6 |
| matrix) | (comp.) | invention | invention | invention | invention | invention |
| Plant Fat | 19.00% | 89.50% | 59.50% | 59.50% | 61.50% | 61.50% |
| (% w wet fatty | | | | | | |
| matrix) | | | | | | |
| including | 60.00% | 60.00% | 35.00% | 60.00% | 92.00% | 92.00% |
| triglycerides | | | | | | |
| (% w plant fat) | | | | | | |
| Beef cells or | 67.50% | 4.00% | 37.00% | 37.00% | 35.00% | 0.00% |
| extracts | | | | | | |
| (% w wet fatty | | | | | | |
| matrix) | | | | | | |
| including beef | 6.75% | 0.40% | 3.70% | 3.70% | 3.50% | 0.00% |
| proteins (% w wet | | | | | | |
| fatty matrix) | | | | | | |
| Transglutaminase | 1.00% | 1.00% | 1.00% | 1.00% | 1.00% | 0.00% |
| Methylcellulose | 0.00% | 0.00% | 0.00% | 0.00% | 0.00% | 1.00% |
| NaCl | 1.00% | 1.00% | 1.00% | 1.00% | 1.00% | 1.00% |
| (% w wet fatty | | | | | | |
| matrix) | | | | | | |
| Flavors (% w wet | 1.50% | 1.50% | 1.50% | 1.50% | 1.50% | 1.50% |
| fatty matrix) | | | | | | |
| Water | 10.00% | 3.00% | 0.00% | 0.00% | 0.00% | 35.00% |
| Total (% w) | 100.00% | 100.00% | 100.00% | 100.00% | 100.00% | 100.00% |
| Texture evaluation | Stable | Good at | Good at | Good at | Good at | Good at |
| | emulsion | 4° C. - | 4° C. - | 4° C. and | 4° C. and | 4° C. and |
| | at 4° C. - | Acceptable | Bad at | 40° C. | 40° C. | 40° C. |
| | Bad | at 40° C. - | 40° C. | | | |
| | texture | | | | | |
| | at 40° C. | | | | | |

### Results and Conclusion

In particular, evaluation based on sensory analysis is conducted on cold (about 400) and after cooking (about 4000) composition. The fatty matrix that is produced in these experiments needs to reproduce an appropriate mouthfeel in cold and hot conditions, mimicking the mouthfeel whilst eating an animal fat product.

The preparation A1 is a comparative (comp.) example. It is not related to the invention. The fatty matrix that can be generated from this composition does not have a texture expected for a meat substitute and cannot be used to produce a protein thread embedded in the fatty matrix, moreover the emulsion is not stable.

Conversely, the compositions A2, A3, A4, and A5 allow to obtain an edible food product comprising protein threads embedded in the fatty matrix, said edible food product having a texture expected for a meat substitute.

In particular, the fatty matrix prepared with the composition A4 produces a food product which may have a better texture at 40° C. compared to the one prepared with the composition A3. Moreover, the food product with the fatty matrix prepared with the composition A5 shows a lower release of fat during cooking compared to the one prepared with A4. In a notable way, a protein-free emulsion can be produced using methylcellulose (composition A6). At the same fat % the texture of the fatty matrix is acceptable when using either protein or methylcellulose as stabilizer (A5 and A6). However, the fat release at 50° C. is higher for the fatty matrix prepared with the composition A6 compared to the one prepared with A5, highlighting the benefit of proteins when present in the fatty matrix.

Fatty Acids Composition of the Fatty Matrix Associated with the Embedded Protein Threads

The fatty acid composition of the fat used in the fatty matrix of the edible product of the invention can have an influence on the stability of the emulsion and the texture obtained for the edible food product comprising protein threads embedded in the fatty matrix.

The protein matrix is prepared by mixing: 17.6% animal proteins from cells or cell extracts wt %; 10% liquid oil; 1% NaCl; 1% sodium alginate; and 70.4% water.

The fatty matrix is prepared by mixing the ingredients following table 2.

The protein matrix is used in a spinneret to induce a flow of the protein matrix coming into contact with the fatty matrix used as a coagulation bath.

Table 2 below shows compositions which solve the technical problem addressed by the present invention.

TABLE 2

| | B1 | B2 | B3 | B4 | B5 | B6 |
|---|---|---|---|---|---|---|
| Ingredients | invention | invention | invention | invention | invention | invention |
| Plant Fat | 65.00% | 65.00% | 65.00% | 65.00% | 65.00% | 65.00% |
| (% w wet fatty matrix) | | | | | | |
| Including Triglycerides | 92.00% | 92.00% | 92.00% | 92.00% | 92.00% | 92.00% |
| (% w plant fat) | | | | | | |
| Palmitic acid | 59.00% | 28.00% | 31.00% | 35.00% | 31.00% | 20.00% |
| (% w plant fat) | | | | | | |
| Stearic acid | 0.00% | 15.00% | 4.00% | 0.00% | 4.00% | 15.00% |
| (% w plant fat) | | | | | | |
| Oleic acid | 33.00% | 52.00% | 58.00% | 55.00% | 55.00% | 55.00% |
| (% w plant fat) | | | | | | |
| Linoleic acid | 5.00% | 1.50% | 2.00% | 5.00% | 5.00% | 5.00% |
| (% w plant fat) | | | | | | |
| Linolenic acid | 0.00% | 0.00% | 1.50% | 2.00% | 2.00% | 2.00% |
| (% w plant fat) | | | | | | |
| Saturated C16-C18 fatty | 59.00% | 43.00% | 35.00% | 35.00% | 35.00% | 35.00% |
| acids | | | | | | |
| (% w plant fat) | | | | | | |
| Monounsaturated C18 | 33.00% | 52.00% | 58.00% | 55.00% | 55.00% | 55.00% |
| fatty acids | | | | | | |
| (% w plant fat) | | | | | | |
| Polyunsaturated C18 fatty | 5.00% | 1.50% | 3.50% | 7.00% | 7.00% | 7.00% |
| acids | | | | | | |
| (% w plant fat) | | | | | | |
| Beef cells or extracts | 32.00% | 32.00% | 32.00% | 32.00% | 32.00% | 32.00% |
| (% w wet fatty matrix) | | | | | | |
| Methylcellulose (% w wet | 0.50% | 0.00% | 0.00% | 0.00% | 0.00% | 0.00% |
| fatty matrix) | | | | | | |
| Transglutaminase | 0.00% | 0.50% | 0.50% | 0.50% | 0.50% | 0.50% |
| (% w wet fatty matrix) | | | | | | |
| Calcium chloride (% w wet | 0.00% | 0.50% | 0.50% | 0.50% | 0.50% | 0.50% |
| fatty matrix) | | | | | | |
| Flavors/colorants | 1.00% | 1.00% | 1.00% | 1.00% | 1.00% | 1.00% |
| (% w wet fatty matrix) | | | | | | |
| Water (% w wet fatty | 1.50% | 1.00% | 1.00% | 1.00% | 1.00% | 1.00% |
| matrix) | | | | | | |
| Total (% w wet fatty matrix) | 100% | 100% | 100% | 100% | 100% | 100% |

The tests carried out from variable concentrations of fats and triglycerides make it possible to assess the importance of the composition on the organoleptic satisfaction brought to the consumer by the protein threads embedded in the fatty matrix. The composition B1 & B2 are less preferred edible food products.

The preparations B5 and B6 give the best results with good results for preparation B3. For the preparation B4, the texture is good at low temperature but just acceptable at high temperature.

### Evaluation of the Effect of the Protein Content in the Protein Matrix on the Food Product According to the Invention

The objective of this experiment is to illustrate the effect of protein content on the mechanical properties of the edible food product through its impact on the protein threads embedded in the fatty matrix.

The protein matrix is prepared by mixing the ingredients following table 3.

TABLE 3
Ingredients of the protein matrix

| | Animal proteins | | | | |
|---|---|---|---|---|---|
| | from cultivated | | Liquid | | Sodium |
| | cells or cell | Water | oil | NaCl | alginate |
| Ref | extracts wt % | wt % | wt % | wt % | wt % |
| C1 | 7.1 | 80.90 | 10 | 1 | 1 |
| C2 | 10.56 | 77.44 | 10 | 1 | 1 |
| C3 | 13.20 | 74.80 | 10 | 1 | 1 |
| C4 | 17.6 | 71.40 | 10 | 1 | 1 |

The fatty matrix is a coagulation bath comprising 70% fat, 2% proteins, 1% transglutaminase, 0.5% w/w of calcium chloride (i.e. 0.18% of the polyvalent ion: calcium), and 26.5% water according to previous examples.

The protein matrix is used in a spinneret to produce a flow of the protein matrix coming into contact with the fatty matrix used as a coagulation bath.

After incubation at 37° C. for 1 h, and before the fatty matrix sets, the mechanical properties of the protein threads embedded in the fatty matrix are evaluated by tensile testing using the texturometer.

### Results & Conclusion

Protein threads embedded in the fatty matrix prepared with the protein matrix having a protein content of 10.56 wt % (C2), 13.20 wt % (C3) or 17.6 wt % (C4) have significant improved strain at break, load at break, and tensile stress compared to the ones having a protein content of 7.1 wt % (C1).

In particular, protein threads embedded in the fatty matrix prepared with the protein matrix having a protein content of 17.6 wt % (C4) have +4% strain at break, +46% load at break, and +54% tensile stress compared to the ones having a protein content of 10.56 wt % (C2).

Hence, when forming protein threads embedded in the fatty matrix according to the invention, one should preferably use a protein matrix having at least 10 wt % of protein compared to the total wet weight of the protein matrix and preferably at least 13 wt %, more preferably at least 17 wt % of protein compared to the wet weight of the protein matrix.

### Evaluation of the Nature of the Texturizing Molecule in the Protein Matrix on the Properties of the Protein Threads

The objective of this experiment is to illustrate the effect of the nature of the texturizing molecule (enzyme or polyelectrolyte) on the mechanical properties of the produced threads. The effect of combining both enzyme and hydrocolloid is also evaluated.

The protein matrix is prepared by mixing the ingredients following table 4.

TABLE 4
Ingredients of the protein matrix

| | Animal | | | | | |
|---|---|---|---|---|---|---|
| | proteins from | | | | | |
| | cultivated | | | | | |
| | cells or cell | | Liquid | | Sodium | Transglu- |
| | extracts | Water | oil | NaCl | alginate | taminase |
| Ref | wt % | wt % | wt % | wt % | wt % | wt % |
| D1 | 17.6 | 69.9 | 10 | 1.5 | 1 | 0 |
| D2 | 17.6 | 69.9 | 10 | 1.5 | 0 | 1 |
| D3 | 17.6 | 68.9 | 10 | 1.5 | 1 | 1 |

The fatty matrix is a coagulation bath comprising 70% fat, 2% proteins, and 1.0% w/w of calcium chloride (i.e. 0.36% of calcium—polyvalent ion), 27% water according to previous examples on fatty matrix composition.

The protein matrix is used in a spinneret to produce the flow of the protein matrix coming into contact with the fatty matrix used as a coagulation bath, then incubated at 37° C. during 1 hour.

### Results

The mechanical properties of the protein threads embedded in the fatty matrix are illustrated in the table 5 below. The results are normalized over the values obtained for protein threads embedded in the fatty matrix made of sodium alginate only (D1).

Protein threads embedded in the fatty matrix can be produced using protein matrices containing alginate or transglutaminase or the combination of both molecules in combination with the fatty matrix. Protein threads prepared with the combination of sodium alginate and transglutaminase (D3) have significantly improved mechanical properties compared to sodium alginate alone (D1) or transglutaminase alone (D2).

TABLE 5

| | Strain at | Load at | Tensile | Young's |
|---|---|---|---|---|
| Ref | break | break | stress | modulus |
| D1 | 100% | 100% | 100% | 100% |
| D2 | 93% | 98% | 91% | 95% |
| D3 | 140% | 133% | 146% | 129% |

### CONCLUSION

Both enzymes (e.g. transglutaminase) and polyelectrolytes (e.g. alginate) under different mechanisms, allow to produce protein threads according to the invention. A combination of both enzyme and polyelectrolyte result in protein threads with improved mechanical properties.

### Evaluation of the Effect of the Nature of the Polyelectrolyte on the Edible Food Product

The objective of this experiment is to determine the effect of the nature of the polyelectrolyte on the edible food product and in particular on the protein threads when embedded with the fatty matrix.

The protein matrix is prepared by mixing the ingredients following table 6.

TABLE 6
Ingredients of the protein matrix (all ingredients excepted
water are expressed in dry weight/total weight of the matrix)

| | Animal | | | | | |
|---|---|---|---|---|---|---|
| | proteins | | | | | |
| | from cells | | Liq- | | | |
| | or cell | | uid | | Transglu- | |
| | extracts | Polyelectrolyte | oil | NaCl | taminase | Water |
| Ref | wt % | wt % | wt % | wt % | wt % | wt % |
| E1 | 16.8 | 1.1 - alginate | 10 | 1.5 | 1 | 69.6 |
| E2 | 16.8 | 1.1 - LMC pectin | 10 | 1.5 | 1 | 69.6 |
| E3 | 16.8 | 1.1 - gelatin 250 | 10 | 1.5 | 1 | 69.6 |
| | | bloom | | | | |
| E4 | 16.8 | 0.8 - chitosan | 10 | 1.5 | 1 | 69.9 |

According to previous examples on fatty matrix composition, the fatty matrix is a coagulation bath comprising 76.5% fat, 2% proteins, 1% transglutaminase, 0.5% w/w of calcium chloride, 20% water; except for D4 where the fatty matrix comprises sodium phosphate dibasic at 1 mol/L instead of calcium chloride.

The protein matrix is used in a spinneret to induce the flow of the protein matrix coming into contact with the fatty matrix used as a coagulation bath.

After incubation at 37° C. for 1 h, the mechanical properties of the edible food product comprising the protein threads embedded in the fatty matrix are evaluated.

### Results & Conclusions

It is possible to form an edible food product comprising heat-resistant protein threads embedded in the fatty matrix with alginate (E1), LMC pectin (E2) and chitosan (E4) whereas gelatin (E3) creates an edible food product comprising protein threads that melt completely when cooking at subsequent step.

The mechanical properties of the protein threads are illustrated in the table 7 below. The results of tensile tests are normalized based on the values obtained with the protein threads made with alginate (E1). Tensile tests were not performed on protein threads prepared with gelatin in the protein matrix (E3) as the protein threads melt when cooking.

TABLE 7

| | Strain at | Load at | Tensile | Young's |
|---|---|---|---|---|
| Ref | break | Break | Stress | modulus |
| E1 | 100% | 100% | 100% | 100% |
| E2 | 101% | 96% | 97% | 93% |
| E4 | 65% | 57% | 69% | 75% |

Hence, either an interaction between a cationic polyelectrolyte with a polyanion or an anionic polyelectrolyte with a polycation works in a method according to the invention. This stresses the efficiency of using a gelation mediated by ionic interaction (quick polymerization when contacting two compositions) to obtain a food product comprising protein threads embedded in the fatty matrix capable of retaining their structure when cooking. This is of first importance to ensure a good acceptance by the consumer who can cook and eat the edible food product from the invention compared to other alternatives of conventional meat products from slaughtered animals.

### Evaluation of the Effect of the Temperature on the Protein Threads

The objective of this experiment is to determine the maximum temperature to which the protein matrix can be subjected before spinning and the effect of high temperatures of the fatty matrix at atmospheric pressure. Indeed, in some embodiments, the fat used in the fatty matrix needs to be heated to obtain an homogeneous matrix. However, the temperature of the matrix can have an impact on the properties of the edible food product.

The protein matrix is prepared by mixing the ingredients following table 8.

TABLE 8
Ingredients of the protein matrix (all ingredients excepted
water are expressed in dry weight/total weight of the matrix)

| | Animal | | | | |
|---|---|---|---|---|---|
| | proteins from | Liquid | | | |
| | cells or cell | oil | NaCl | Alginate | Water |
| Ref | extracts wt % | wt % | wt % | wt % | wt % |
| F | 12.6 | 15 | 1 | 1 | 70.4 |

The fatty matrix is a coagulation bath comprising 80.5% fat, 8% proteins, 1% transglutaminase, 0.5% w/w of calcium chloride (0.18% of calcium—polyvalent ion) and 10% water according to previous examples on fatty matrix composition.

The protein matrix is used in a spinneret to induce the flow of the protein matrix coming into contact with the fatty matrix used as a coagulation bath.

In order to evaluate the effect of the temperature of the protein matrix on the method of the invention, the protein matrix is incubated for 10 minutes at different temperatures: at room temperature (RT), 55° C., 60° C., or 65° C. The protein matrix is used in a spinneret to induce the flow of the protein matrix coming into contact with the fatty matrix used as a coagulation bath, being at room temperature (about 20° C.).

In order to evaluate the effect of the temperature of the fatty matrix as the coagulation bath on the method of the invention, the fatty matrix is incubated for 10 minutes at: 65° C., 75° C. and 85° C. and the protein threads embedded in the fatty matrix are produced by contacting the protein matrix (at room temperature) and the fatty matrix, a calcium coagulation bath comprising 60% fat.

The protein threads are incubated for 1 minute in the fatty matrix before recovery of the food product which consists of the protein threads embedded in the fatty matrix. After recovery, the quality of the food product is evaluated.

### Results & Conclusions

The spinning is possible when the protein matrix is at 55° C. and 60° C., but the protein thread morphology is not as homogeneous as at RT, some lumps and irregularities can be observed on the protein threads' surface after entering the fatty matrix.

When the protein matrix is incubated at 65° C., the number of lumps increases considerably creating non-homogenous protein threads. Hence, the protein matrix should not undergo a heat treatment of 65° C. or more for 10 me.

Regarding the fatty matrix, food products produced with coagulation baths at 65° C. and 75° C. comprise protein threads having a similar appearance to the ones obtained at RT (no lumps) but the color is slightly faded. Protein threads formed in a fatty matrix at 85° C. are acceptable but start having a lumpy texture on the outside.

As it has been described, the method and device according to the invention can be used to produce an edible food product illustrated in FIG. 3.

### Evaluation of the Effect of the Protein Origin in the First Composition on the Protein Threads

The objective of this experiment is to evaluate the effect of the protein origin on the mechanical properties of protein threads using tensile testing.

The first composition is prepared by mixing the ingredients following table 9a.

TABLE 9a
Ingredients of the first composition (all ingredients except for
water are expressed in weight/total weight of the composition)

| | | | B0 |
|---|---|---|---|
| Ref | B80 | B40 | (comparative exp.) |
| Animal proteins from cells or cell | 17.6 | 8.8 | 0 |
| extracts wt % | | | |
| Including myofibrillar proteins wt % | 8.8 | 4.4 | 0 |
| Plant proteins wt % | 0 | 8.8 | 17.6 |
| Proteins total (wt %) | 17.6 | 17.6 | 17.6 |
| Myofibrillar protein on total proteins | 0.5 | 0.25 | 0 |
| Liquid oil wt % | 10 | 10 | 10 |
| NaCl wt % | 1 | 1 | 1 |
| Sodium alginate wt % | 1 | 1 | 1 |
| Water wt % | 70.4 | 70.4 | 70.4 |

The plant proteins used in the first composition are soy proteins. The protein threads are formed by spinning.

After incubation at 37° C. for 1 h, the mechanical properties of the protein threads are evaluated by tensile testing using the texturometer.

### Results & Conclusion

The mechanical properties of the protein threads are illustrated in the table 9b below.

TABLE 9b
Results from the tensile testing

| | Strain at | Load at | Tensile | Young's |
|---|---|---|---|---|
| Ref | break | Break | Stress | modulus |
| B80 | 226% | 309% | 302% | 123% |
| B40 | 213% | 189% | 184% | 105% |
| B0 | 100% | 100% | 100% | 100% |

The results of the tensile tests are normalized over the values obtained for the protein threads made with only plant protein (B0).

Protein threads prepared with animal proteins from cells or cell extracts (B80; B40) have higher resistance to traction (tensile testing) than the ones prepared with soy proteins in the same conditions. The observed improvement is a factor of 2 when the animal proteins from cells or cell extracts (B40) are used in combination with plant proteins (resulting in a concentration of myofibrillar proteins above 4%) and at least a factor 3 when a concentration above 15% of animal proteins from cells or cell extracts is used in weight (B80).

Further, in a method of the invention, the combination of animal proteins from cultivated cells with plant proteins improves the tensile properties of the threads compared to plant proteins alone (B40 compared to B0).

This illustrates that protein threads made according to the method of the invention have desired mechanical properties far above the ones made of plant proteins only.

Moreover, adding some plant proteins can be used to tune the viscoelasticity or flow properties of the first composition comprising non-human animal cultivated cells.

### Texture of an Edible Food Product According to the Invention

Texture is among the main parameters for consumer acceptance of food products. However, the reproduction of a meat texture, in particular from cultured cells, is complex.

Hence, the texture profile analysis (TPA) has been conducted to compare the texture of an edible meat substitute comprising protein threads embedded into the fatty matrix according to the invention to the texture of two commercial food products: a conventional beef and a plant-based substitute both with marbled fat.

Table 10 below shows the TPA characteristics of a conventional beef with marbling (Sample G1), a plant-based substitute (Sample G2), and a composition according to the invention (Sample G3).

TABLE 10

| | | Hardness | Hardness | Cohe- | |
|---|---|---|---|---|---|
| | | 1 | 2 | siveness | Fracture |
| Samples | | (N) | (N) | [TPA] | (N) |
| Sample G1 | Mean | 121.78 | 85.47 | 0.18 | Not |
| (Comp.) | | | | | detected |
| | Standard | 25.73 | 18.31 | 0.02 | Not |
| | deviation | | | | detected |
| Sample G2 | Mean | 88.76 | 58.28 | 0.29 | 8.61 |
| (Comp.) | Standard | 9.18 | 10.66 | 0.09 | 1.72 |
| | deviation | | | | |
| Sample G3 | Mean | 115.52 | 80.34 | 0.16 | Not |
| (Invention) | | | | | detected |
| | Standard | 5.93 | 4.61 | 0.03 | Not |
| | deviation | | | | detected |

As illustrated in this table 10, the food product according to the present invention (Sample G3) has similar texture properties compared to the conventional food product (Sample G1). Additionally, the invention has better performance than the sample G2, which is a plant-based meat substitute.

In particular, an edible food product according to the invention (Sample G3) has a cohesiveness that is significantly closer to the conventional product (Sample G1) compared to the cohesiveness of a plant-based substitute (G2).

Interestingly, there are no fracture detected for the edible food product according to the invention (Sample G3) and the conventional product (Sample G1) while fracture values of 8.61±1.72 N are measured for the plant-based substitute (Sample G2). Visually, the protein threads on the plant-based substitute are completely separated after the first compression while the structure remind cohesive with no separation between the edible protein threats and the fatty matrix, for the edible food product according to the invention, similarly to the conventional product.

Hence, an edible food product comprising edible protein threads embedded into a fatty matrix according to the invention can have a texture similar and comparable to conventional meat.

### Fat Release Properties of an Edible Food Product According to the Invention

As it has been mentioned, the fat release phenomenon during cooking and chewing is important when evaluating the textural properties of meat products, especially high fat content meat products such as marbled beef. Also, the fat release phenomenon can be correlated to the fat-explosion and mouthfeel that consumers experience while chewing.

As an edible food product according to the invention comprises edible protein threads embedded into a fatty matrix, a way of evaluating its ability to mimic the behavior of a conventional meat product is to evaluate the quantity of leaked fat during a pan-fried style cooking.

Table 11 below shows the results of fat release measurements of a conventional beef with marbling (Sample G1) and a plant-based substitute (Sample G2), and a composition according to the invention (Sample G3).

TABLE 11

| | | Fat released | Fat released |
|---|---|---|---|
| | | during | during |
| | | cooking | compression |
| Samples | | (w/w %) | (w/w %) |
| Sample G1 | Mean | 8.31 | 10.67 |
| (Comp.) | Standard deviation | 2.29 | 2.35 |
| Sample G2 | Mean | 4.22 | 2.47 |
| (Comp.) | Standard deviation | 1.05 | 0.51 |
| Sample G3 | Mean | 10.02 | 10.50 |
| (Invention) | Standard deviation | 0.65 | 0.44 |

As illustrated in this table 11, the food product according to the present invention (Sample G3) has similar fat release properties compared to the conventional food product (Sample G1). Indeed, whereas the plant-based substitute (Sample G2) will only leak around 4% of its initial weight, a product according to the invention will leak more than 9% during cooking.

The results also show that the product according to the invention has a behavior similar to conventional meat with marbled fat even during cooking. Moreover, an edible food product comprising edible protein threads embedded into a fatty matrix according to the invention has a cooking behavior similar to the cooking behavior of a conventional meat product and thus reproduces for the consumer the experience of cooking conventional food products.

Under conditions of thermal and mechanical stress, such as those occurring when cooking and when chewing, fat release during compression results show that a product comprising edible protein threads embedded into a fatty matrix according to the invention will behave as a conventional food product.

Also, such behavior can be correlated to the fat-explosion that the consumer experiences in the mouth when eating marbled beef cuts.

Hence, this fat release measurements show that an edible food product comprising edible protein threads embedded into a fatty matrix according to the invention has a texture similar to conventional meat.

Thus, in general, a food product according to the invention has a texture before cooking, while cooking, after cooking and while chewing, similar to a conventional meat. The food product according to the present invention exhibits overall qualities close to those of conventional meat and matches the consumer experience when cooking and eating conventional meat.

### Sensory Evaluations of the Appearance and the Texture of Edible Food Products

The products according to the invention showed textural properties similar to those of conventional products. To further evaluate organoleptic properties of food products according to the invention, sensory evaluations were conducted on a conventional marbled beef (Sample G1), a plant-based meat substitute (Sample G2) and a composition according to the invention (Sample G3). The sensory evaluations included 20 trained panelists.

As a result, the edible food product (Sample G3) was significantly more similar to the conventional meat product (Sample G1) than the plant-based meat substitute (Sample G2) on all evaluated characteristics (tenderness, cohesiveness, oiliness and juiciness).

Hence, an edible food product comprising edible protein threads embedded into a fatty matrix according to the present invention has similar organoleptic properties to conventional meat.

The invention can be the subject of numerous variants and applications other than those described above. In particular, unless otherwise indicated, the different structural and functional characteristics of each of the implementations described above should not be considered as combined and/or closely and/or inextricably linked to each other, but on the contrary as simple juxtapositions. In addition, the structural and/or functional characteristics of the various embodiments described above may be the subject in whole or in part of any different juxtaposition or any different combination.

1. Method of manufacturing an edible food product comprising edible protein threads embedded in a fatty matrix, said method comprising the following steps:
Preparing a protein matrix, said protein matrix comprising proteins,
Preparing a fatty matrix, said fatty matrix comprising at least 20% in weight of fat compared to a total weight of the fatty matrix,
the protein matrix and/or the fatty matrix comprising one or more texturizing molecule(s) capable of creating a heat-resistant thread, and
Contacting the protein matrix with the fatty matrix to form edible protein threads, from the protein matrix, embedded into the fatty matrix.

2. Method according to claim 1, wherein said fatty matrix further comprises at least 0.25% in weight of non-human animal proteins with respect to a total wet weight of the fatty matrix.

3. Method according to claim 1, wherein said fatty matrix comprises triglycerides and the triglycerides of the fatty matrix comprise more than 1.5% in weight of polyunsaturated C18 fatty acids with respect to a total weight of triglycerides in the fatty matrix.

4. Method according to claim 1, wherein said fatty matrix comprises triglycerides and the triglycerides of the fatty matrix comprise more than 0.01% in weight of linolenic acids with respect to a total weight of triglycerides in the fatty matrix.

5. Method according to claim 1, wherein said protein matrix comprises at least 0.5% in weight of animal proteins being from non-human cultivated animal cells compared to a total wet weight of the protein matrix.

6. Method according to claim 1, wherein said protein matrix comprises at least 5% in weight of animal proteins being from non-human cultivated animal cells compared to a total weight of proteins in the protein matrix.

7. Method according to claim 1, wherein said protein matrix comprises at least 0.25% in weight of myofibrillar proteins compared to a total weight of proteins.

8. Method according to claim 1, wherein said protein matrix comprises at least 5% in weight of proteins compared to a total weight of the protein matrix.

9. Method according to claim 1, wherein said texturizing molecule(s) comprises a combination of a polyvalent ion and a polyelectrolyte, said polyelectrolyte being capable of complexing with the polyvalent ion to form a heat-resistant complex.

10. Method according to claim 1, wherein the fat of the fatty matrix comprises at least 60% in weight of triglycerides compared to a total weight of the fat of the fatty matrix.

11. Method according to claim 1, wherein the fat of the fatty matrix comprises at least 20% in weight of unsaturated fatty acids compared to a total weight of the fat of the fatty matrix.

12. Method according to claim 1, wherein the preparation step of the fatty matrix further comprises a homogenizing step.

13. Method according to claim 1, wherein the fatty matrix comprises an oil in water emulsion.

14. Method according to claim 1, wherein the fatty matrix further comprises at least 0.25% in weight of proteins compared to a total weight of the fatty matrix.

15. Method according to claim 1, wherein the contacting step comprises an injection of the protein matrix into the fatty matrix at an injection site, and a movement of the injection site of the protein matrix into the fatty matrix relative to the fatty matrix.

16. Method according to claim 1, wherein the preparation step of the fatty matrix further comprises a heat treatment of the fat to be used in the fatty matrix.

17. Method according to claim 1, wherein the contacting step is conducted at a temperature of at least 30° C. and the contacting step is followed by a cooling step of the protein threads embedded in the fatty matrix.

18. Method according to claim 1, wherein the fatty matrix comprises:
at least 20% in weight of fat with respect to a total wet weight of the fatty matrix;
at least one said texturizing molecule; and
proteins, said proteins comprising non-human animal proteins from non-human cultivated animal cells.

19. Edible food product obtained from the method according to claim 1, said edible food product comprising edible protein threads embedded in said fatty matrix, said edible protein threads being heat-resistant threads comprising proteins and the fatty matrix comprising at least 20% in weight of fat compared to a total weight of the fatty matrix.

20. Edible food product according to claim 19 wherein the proteins of the edible protein threads comprise animal proteins from non-human cultivated animal cells.

21. System for producing an edible food product comprising edible protein threads embedded in a fatty matrix, said system comprising:
A protein matrix container capable of containing a protein matrix, said protein matrix comprising proteins,
A fatty matrix container capable of containing a fatty matrix, said fatty matrix comprising at least 20% in weight of fat compared to a total weight of the fatty matrix, the protein matrix and/or the fatty matrix comprising one or more texturizing molecule(s) capable of creating a heat-resistant thread, and
A contacting device configured to bring in contact the protein matrix with the fatty matrix to form, from the protein matrix, edible protein threads embedded into the fatty matrix, said contacting device being arranged to inject the protein matrix into the fatty matrix in at least one injection site;
said system being configured to induce a movement of:
the at least one injection site of the protein matrix into the fatty matrix relative to the fatty matrix, and/or
the fatty matrix relative to the at least one injection site of the protein matrix into the fatty matrix.
